(19) United States
(12) Patent Application Publication
(10) Pub. No.: US 2025/0192191 A1
(43) Pub. Date: Jun. 12, 2025

(54) ELECTRON CATALYZED MOLECULAR RECOGNITION

(71) Applicant: Northwestern University, Evanston, IL (US)

(72) Inventors: Yang Jiao, Evanston, IL (US); Yunyan Qiu, Evanston, IL (US); James Fraser Stoddart, Evanston, IL (US)

(21) Appl. No.: 18/844,941

(22) PCT Filed: Mar. 8, 2023
(86) PCT No.: PCT/US2023/063994

§ 371 Date: Sep. 6, 2024

Related U.S. Application Data
(60) Provisional application No. 63/317,849, filed on Mar. 8, 2022.

(51) Int. Cl. H01M 4/90; B01J 35/80

(52) U.S. Cl. CPC H01M 4/90; B01J 35/80

(57) ABSTRACT

Disclosed herein are systems for electron catalyzed molecular recognition and methods of making and using the same. The system comprises an electron source for providing an electron, a redox-active substrate capable of accepting the electron from the electron source, and a catalytic intermediate formed noncovalently from the substrate and a second molecule, wherein the energy barrier for forming the catalytic intermediate is decreased by the redox-active substrate accepting the electron from the electron source.

### CROSS-REFERENCE TO RELATED APPLICATIONS

The present application claims priority to U.S. Provisional Patent Application No. 63/317,849, filed Mar. 8, 2022, the entire contents of which are hereby incorporated by reference.

### FIELD OF THE INVENTION

The disclosed technology is generally directed to methods for supramolecular assembly. More particularly the technology is directed to molecular recognition by electron catalysis.

### BACKGROUND OF THE INVENTION

Molecular recognition and supramolecular assembly cover a broad spectrum of noncovalently orchestrated phenomena between molecules. Catalysis of such processes have been limited to ones that rely on sophisticated catalyst design. As a result, there is a need for versatile strategies to facility molecular recognition into the realm of supramolecular noncovalent chemistry by catalysis.

### BRIEF SUMMARY OF THE INVENTION

Disclosed herein are systems for electron catalyzed molecular recognition and methods of making and using the same. The system comprises an electron source for providing an electron, a redox-active substrate capable of accepting the electron from the electron source, and a catalytic intermediate formed noncovalently from the substrate and a second molecule, wherein the energy barrier for forming the catalytic intermediate is decreased by the redox-active substrate accepting the electron from the electron source.

Another aspect of the technology provides for a method for electron catalyzed molecular recognition. The method comprises providing, with an electron source, an electron to a redox-active substrate capable of accepting the electron from the electron source; and forming noncovalently a catalytic intermediate from the redox-active substrate and a second molecule, wherein the energy barrier for forming the catalytic intermediate is decreased by the redox-active substrate accepting the electron from the electron source.

These and other aspects of the technology will be further described herein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1: Design of an electron-catalysed molecular recognition process. a, Structural formulae and graphical representations of the host-guest system, which includes a macrocyclic host, R^{2(•+)} and a dumbbell-shaped guest, D^{+(•+)}, investigated in this research. Electron catalysis arises from the fact that each bipyridinium (BIPY) unit in the system has three redox states, i.e., dicationic, radical cationic and neutral states. b, Proposed mechanism for an electron-catalysed molecular recognition process. The direct (grey dashed arrow) formation of a trisradical complex [D⊂R]^{+3(•+)} from R^{2(•+)} and D^{+(•+)} is all but kinetically forbidden. The catalytic (black solid arrows) complexation comprises four steps, including one initiating step and three propagating steps. Step 1: the injection of an electron. Step 2: a single-electron reduction of either one of the BIPY^{•-} units in R^{2(•+)} or the BIPY^{•+} unit in D^{+(•+)}. Step 3: the rapid formation of a bisradical complex [D⊂R]^{+2(•+)}, the key intermediate, favoured by a decrease in the Coulombic repulsion between the host and guest molecules. Step 4: the oxidation of the bisradical complex [D⊂R]^{+2(•+)}, affording a trisradical complex [D⊂R]^{+3(•+)} as the final product, while releasing an electron to close the catalytic cycle. Notably, although the electron is formally written into the catalytic cycle in order to highlight its role as a catalyst, such a representation does not imply the existence of a free electron during the catalytic process.

FIG. 2: Molecular recognition accelerated by catalytic amounts of cobaltocene (CoCp₂). a-c, The evolution of UV/Vis/NIR spectra within 70 min after combining equimolar amounts of R^{2(•+)} and D^{+(•+)} without added CoCp₂ (a) and with either 4 mol % CoCp₂ (b) or 8 mol % CoCp₂ (c). d, Kinetic traces for the host-guest complexation between R^{2(•+)} and D^{+(•+)} in the presence of different amounts of CoCp₂, obtained by plotting the yield of the [D⊂R]^{+3(•+)} trisradical complex against time. The concentrations of R^{2(•+)} and D^{+(•+)} were both fixed at 150 μM, and the concentration of CoCp₂ was set to be 0, 1.5, 3, 6 and 12 μM in different experiments.

FIG. 3: Detection and verification of the key intermediate in the electron-catalysed molecular recognition process. a-b, A stepwise electron cycle monitored by UV/Vis/NIR spectroscopy. Initial state: Molecular recognition between R^{2(•+)} and D^{+(•+)} occurs to a negligible extent (b). Electron injection: 1 Molar equivalent of CoCp₂ was introduced into the system, leading to the formation of the [D⊂R]^{+2(•+)} bisradical complex (b). Electron removal: Excess of either R^{2(•+)} or D^{+(•+)} was introduced to oxidize the bisradical complex to the [D⊂R]^{+3(•+)} trisradical complex (blue line in b). c-f, Investigation of a [2]catenane (Cat⁶⁺) model compound, focusing on its bisradical dicationic state, Cat^{2(•+)}. c, Combined structural formulae and graphical representation of the quantitative transformation from Cat^{3(•+)} to Cat^{2(•+)} using 1 molar equivalent of CoCp₂. d, Evolution of UV/Vis/NIR spectra during the titration with CoCp₂, and photographs showing the colour change in the MeCN solution as Cat^{3(•+)} is reduced to Cat^{2(•+)}. The discontinuities in the spectra at around 1700 nm result from the cropping out of the sharp solvent peaks to present clearly the absorption of Cat^{2(•+)}. e, Electron paramagnetic resonance (EPR) spectra of Cat^{3(•+)} and Cat^{2(•+)} recorded in MeCN at room temperature. f, Single-crystal structure of Cat^{2(•+)}, with annotated centroid-to-centroid distances between adjacent BIPY^{•+/(0)} units. Hydrogen atoms are omitted for the sake of clarity.

FIG. 4: Electrochemically controlled molecular recognition. a, A combined structural formulae and graphical representation of one of the possible pathways involving molecular recognition during the electrolysis in an undivided cell. The overall process is composed of three stages. (1) Initiation: Upon applying electricity, one BIPY^{•+} unit in an R^{2(•+)} is reduced to BIPY^{(O)} at the cathode. Concurrently, one BIPY^{•-} unit in another R^{2(•+)} is oxidized to BIPY²⁺ at the anode. (2) Propagation: The resulting R^{•+} binds rapidly with D^{+(•+)} to form a [D⊂R]^{+2(•+)} bisradical complex. This intermediate undergoes single-electron transfer (SET) with R^{2(•+)}, affording a [D⊂R]^{+3(•+)} trisradical complex as the final product and generating a new R to restart the cycle. (3) Termination: The [D⊂R]^{+2(•+)} bisradical complex transfers an electron to R^{2+(•+)}, recovering R^{2(•+)} as the starting material, or undergoes a back electron transfer (BET) to the anode and generates a [D⊂R]^{+3(•+)} trisradical complex. b-c, Changes in the absorbance at 1080 nm during the intermittent electrolysis of the mixture of R^{2(•+)} and D^{+(•+)} under different conditions. b, Stirring rate was fixed at 300 rpm, and current intensity was set to be 0.5, 1.0 and 2.0 mA. c, Current intensity was fixed at 1.0 mA, and stirring rate was set to be 200, 300 and 400 rpm.

FIG. 5: The electron as an efficient catalyst for both covalent reactions and molecular recognition. a, Electron-catalysed covalent reactions are well established in synthetic covalent chemistry, particularly for the radical-mediated photoredox catalysis and organic electrosynthesis. Consider a reaction A+B→A−B, where a high energy barrier causes the reaction to be very slow. Injection of an electron reduces one of the substrates (A) to a highly reactive radical species (A^{•-}), which can rapidly form a covalent bond with the other substrate (B). The resulting intermediate (A^{•-}-B) releases the electron to afford the final product (A-B). Whereas the overall process is redox neutral, i.e., the redox state remains the same during the transformation from the substrates to product, the catalytic pathway, which involves the temporary addition of an electron, leads to a substantially lower energy barrier, thereby expediting the formation of the covalent bond. In this process, the electron has acted as an effective catalyst. b, The research reported in this article aims to extend the paradigm of electron catalysis to promoting and controlling molecular recognition. The trajectory for this noncovalent process is similar to that for electron-catalysed covalent reactions, except that the product is a supramolecular complex wherein molecular components are assembled courtesy of noncovalent bonding interaction(s), rather than a molecule whose atoms are connected by covalent bond(s).

FIG. 6: ¹H-¹H COSY spectrum (500 MHz, CD₃CN, 298 K) of D•3PF₆

FIG. 7: ¹H-¹H COSY spectrum (500 MHz, CD₃CN, 298 K) of Cat•6PF₆

FIG. 8: Structural formulae and graphical representations of the chemical entities (the macrocyclic host and the dumbbell-shaped guest molecules) investigated in this research.

FIG. 9: Structural formulae and graphical representations of the chemical entities (the host-guest complexes and the [2]catenane) investigated in this research.

FIG. 10: a) Controlled potential electrolysis (CPE) of the BIPY²⁺ unit(s) in R⁴⁺ or D³⁺ to produce BIPY^{•+} radical cations. In order to avoid the over-reduction, the working potentials for CPE were determined according to the CV and DPV data of b) R⁴⁺ and c) D³⁺.

FIG. 11: a) The evolution of UV/Vis/NIR spectra within 10 h after combining equimolar amounts of R^{2(•+)} and D^{+(•+)}. b) Time-dependent changes of the absorbances at 600 nm and 1080 nm during the process.

FIG. 12: a) The evolution of UV/Vis/NIR spectra within 10 h after combining equimolar amounts of R^{2(•+)} and D^{+(•+)} with the addition of 20 mol % NOPF₆. b) Time-dependent changes of the absorbances at 600 nm and 1080 nm during the process.

FIG. 13: Linear fitting of the kinetic data to determine the rate constants of the molecular recognition between R^{2(•+)} and D^{+(•+)} in the presence of a) no additive, b) 20 mol % NOPF₆, c) 1 mol % CoCp₂, d) 2 mol % CoCp₂, e) 4 mol % CoCp₂ and f) 8 mol % CoCp₂.

FIG. 14: CV data for a) R⁴⁺, b) D³⁺ and c-e) the equimolar mixture of R⁴⁺ and D³⁺ with different scan ranges: from +0.5 to −1.2 V (c), from +0.5 to −0.75 V (d) or from +0.5 to −0.6 V (e). The data were recorded at room temperature and the concentrations of all the samples were set to 1.0 mM. Each measurement includes three cycles of scan and the figure shows the first cycle.

FIG. 15: CV data for the mixture of R⁴⁺ and D³⁺ at different scan rates of 0.01, 0.02, 0.05, 0.1, 0.2, 0.5, 1, 2, 5, 10, 20 and 50 V/s. The concentrations of both compounds were 1.0 mM in MeCN solution. Each measurement includes three cycles of scan and the figure shows the first cycle.

FIG. 16: The first and second cycles of the CV scan on the equimolar mixture of R⁴⁺ and D³⁺, as well as the graphical representation for the first reduction peak (−63 mV) in the second cycle. The concentrations of both compounds were 1.0 mM in MeCN solution. The scan rate was set to 100 mV s⁻¹.

FIG. 17: The calculated potential energy surface associated with the binding process between R^{2(•+)} and D^{+(•+)}. The value of the energy barrier (ΔE^{‡}) was determined by the energy difference between the position 1 and 4. The QM calculations were done with a net charge of +2, with two Cl⁻ counterions included.

FIG. 18: The calculated potential energy surface associated with the binding process between R^{•+} and D^{+(•+)}. The value of the energy barrier (ΔE^{‡}) was determined by the energy difference between the position 3 and 7. The QM calculations were done with a net charge of +1, with two Cl⁻ counterions included.

FIG. 19: The calculated potential energy surface associated with the binding process between R^{2(•+)} and D⁺. The value of the energy barrier (ΔE^{‡}) was determined by the energy difference between the position 3 and 5. The QM calculations were done with a net charge of +1, with two Cl⁻ counterions included.

FIG. 20: Comparison of the potential energy surface for the molecular recognition between R^{2(•+)} and D^{+(•+)} with or without injection of an electron.

FIG. 21: UV/Vis/NIR spectra of the equimolar mixture of R^{2(•+)} and D^{+(•+)} after adding different amounts (0, 4, 20, 50 and 100 mol %) of CoCp₂. The spectra were recorded after the systems reached kinetically steady state (without the addition of CoCp₂) or thermodynamic equilibrium (with the addition of CoCp₂). The discontinuities at around 1700 nm result from the cropping out of the sharp solvent peaks.

FIG. 22: The combination of R^{•+} and D^{+(•+)} leads to the instantaneous formation of the [D⊂R]^{+2(•+)} bisradical complex, as indicated by the UV/Vis/NIR spectra of R^{•+}, D^{+(•+)} and their equimolar mixture. R^{•+} was prepared by adding 1 eq CoCp₂ into the solution of R^{2(•+)}, and then mixed with D^{+(•+)}.

FIG. 23: The combination of R^{2(•+)} and D⁺ leads to the instantaneous formation of the [D⊂R]^{+2(•+)} bisradical complex, as indicated by the UV/Vis/NIR spectra of R^{2(•+)}, D⁺ and their equimolar mixture. D⁺ was prepared by adding 1 eq CoCp₂ into the solution of D^{+(•+)}, and then mixed with R^{2(•+)}.

FIG. 24: The experimental evidence for the SET between the [D⊂R]^{+2(•+)} bisradical complex and R^{2(•+)}. a) UV/Vis/NIR spectra of the solution of [D⊂R]^{+2(•+)} bisradical complex before and after the addition of 1 eq R^{2(•+)}. b) The evolution of the difference between the absorbances at 1080 and 1700 nm along with the increased amounts of R^{2(•+)}.

FIG. 25: The experimental evidence for the SET between the [D⊂R]^{+2(•+)} bisradical complex and D^{+(•+)}. a) UV/Vis/NIR spectra of the solution of [D⊂R]^{+2(•+)} bisradical complex before ([D⊂R]^{+2(•+)}) and after ([D⊂R]^{+3(•+)} the addition of 2 eq D^{+(•+)}. b) The evolution of the difference between the absorbances at 1080 and 1700 nm along with the increased amounts of D^{+(•+)}.

FIG. 26: Comparison of the complexity between the host-guest (supramolecular) system and the [2]catenane (molecular) system.

FIG. 27: The transformation between Cat^{3(•+)} and Cat^{2(•+)}, indicated by the evolution of the UV/Vis/NIR spectra during a) the reduction of Cat^{3(•+)} to Cat^{2(•+)} using 1 eq CoCp₂, and b) the oxidation of Cat^{2(•+)} back to Cat^{2(•+)} using excess of R^{2(•+)}. The discontinuities in the spectra at around 1700 nm result from the cropping out of the sharp solvent peaks to present clearly the absorption of Cat^{2(•+)}.

FIG. 28: Different views of the X-ray crystal structure of Cat^{2(•+)}, showing a) the inclusion structure, wherein the R ring encircles the BIPY unit belonging to the other ring. b) the dihedral angle between the BIPY units from two component rings. c) the distances between adjacent BIPY units. Hydrogen atoms and PF₆⁻ counterions are omitted for the sake of clarity.

FIG. 29: Extended superstructures of [Cat•CoCp₂]•3PF₆. Co⁺Cp₂ the oxidation product of CoCp₂—co-crystalise with Cat^{2(•+)}, surrounded by three PF₆⁻ counterions. Neither radical-pairing nor [π . . . π] stacking interactions are found between adjacent molecules. Hydrogen atoms are omitted for the sake of clarity.

FIG. 30:| Summary of the chemical initiators screened in this research. The values of their reduction potentials (versus saturated calomel electrode) are listed in the brackets, in which the reduction potentials of D⁺, R⁽⁰⁾ and Cat^{2(•+)} are measured by CV experiments, while the reduction potentials of other initiators are reported³⁰⁻³² in the literature.

FIG. 31: Comparison of the reduction potentials of the chemical initiators. The values of the reduction potentials are versus saturated calomel electrode (SCE). The dashed line represents the reducing power required for triggering the electron-catalysed molecular recognition between R^{2(•+)} and D^{+(•+)} FIG. 32: The molecular recognition initiated by D⁺. a) The evolution of UV/Vis/NIR spectra within 70 min after combining equimolar amounts of R^{2(•+)} and D^{+(•+)} with 4 mol % D⁺. b) The kinetic trace of the process.

FIG. 33: The molecular recognition initiated by R⁽⁰⁾. a) The evolution of UV/Vis/NIR spectra within 70 min after combining equimolar amounts of R^{2(•+)} and D^{+(•+)} with 2 mol % R⁽⁰⁾. b) The kinetic trace of the process.

FIG. 34: The molecular recognition initiated by Cat^{2(•+)}. a) The evolution of UV/Vis/NIR spectra within 70 min after combining equimolar amounts of R^{2(•+)} and D^{+(•+)} with 4 mol % Cat^{2(•+)}. b) The kinetic trace of the process.

FIG. 35: The molecular recognition initiated by CoCp₂. a) The evolution of UV/Vis/NIR spectra within 70 min after combining equimolar amounts of R^{2(•+)} and D^{+(•+)} with 4 mol % CoCp₂. b) The kinetic trace of the process.

FIG. 36: The molecular recognition initiated by Co(Cp*)₂. a) The evolution of UV/Vis/NIR spectra within 70 min after combining equimolar amounts of R^{2(•+)} and D^{+(•+)} with 4 mol % Co(Cp*)₂. b) The kinetic trace of the process.

FIG. 37: The molecular recognition initiated by TDAE. a) The evolution of UV/Vis/NIR spectra within 70 min after combining equimolar amounts of R^{2(•+)} and D^{+(•+)} with 4 mol % TDAE. b) The kinetic trace of the process.

FIG. 38: The molecular recognition initiated by different metals. The UV/Vis/NIR spectra before and after stirring the equimolar mixture of R^{2(•+)} and D^{+(•+)} for 20 min in the presence of a) Mg dust, b) Al dust, c) Fe dust, d) Zn dust, e) Cu dust, and f) no added metals.

FIG. 39: a) The photograph of a divided electrochemical cell used for initiating electron-catalysed molecular recognition. b) The UV/Vis/NIR spectra before and after the injection of 100 mC of electrons into the equimolar mixture of R^{2(•+)} and D^{+(•+)}.

FIG. 40: a) The evolution of UV/Vis/NIR spectra within 200 min after injecting 25 mC of electrons into the equimolar mixture of R^{2(•+)} and D^{+(•+)}. b) The kinetic trace of the process.

FIG. 41: The first possible pathway involving the molecular recognition during the electrolysis in an undivided cell. In the initiating step, R^{2(•+)} is reduced to R^{•+} at the cathode, while another R^{2(•+)} is oxidized to R^{2+(•+)} at the anode.

FIG. 42: The second possible pathway involving the molecular recognition during the electrolysis in an undivided cell. In the initiating step, R^{2(•+)} is reduced to R at the cathode, while D^{+(•+)} is oxidized to D³⁺ at the anode.

FIG. 43: The third possible pathway involving the molecular recognition during the electrolysis in an undivided cell. In the initiating step, D^{+(•+)} is reduced to D⁺ at the cathode, while R^{2(•+)} is oxidized to R^{2+(•+)} at the anode.

FIG. 44: The fourth possible pathway involving the molecular recognition during the electrolysis in an undivided cell. In the initiating step, D^{+(•+)} is reduced to D⁺ at the cathode, while another D^{+(•+)} is oxidized to D³⁺ at the anode.

FIG. 45: The evolution of UV/Vis/NIR spectra during the intermittent electrolysis of the equimolar mixture of R^{2(•+)} and D^{+(•+)} at 300 rpm stirring rate and a) 0.5, b) 1.0 or c) 2.0 mA current. d) Time-dependent changes in the absorbance at 1080 nm during the electrolysis under different conditions, showing the positive correlation between the kinetics of molecular recognition and the current intensity.

FIG. 46: The evolution of UV/Vis/NIR spectra during the intermittent electrolysis of the equimolar mixture of R^{2(•+)} and D^{+(•+)} at 1.0 mA current and a) 200, b) 300 or c) 400 rpm stirring rate. d) Time-dependent changes in the absorbance at 1080 nm during the electrolysis under different conditions, showing the negative correlation between the kinetics of molecular recognition and the stirring rate.

FIG. 47: A scheme showing the reversible host-guest complexation can be written as the equation given.

FIG. 48: A scheme showing the kinetic model of a two-to-one reversible reaction used to deduce the rate equation.

FIG. 49: Scheme 4 shows a graphic representation of Stage 1 (from +0.5 to −0.6 V) of the CV scan on the mixture of R⁴⁺ and D³⁺.

FIG. 50: Scheme 5 shows a graphic representation of Stage 2 (from −0.6 to −1.2 V) of the CV scan on the mixture of R⁴⁺ and D³⁺.

FIG. 51: Scheme 6 shows a graphic representation of Stage 3 (from −1.2 to −0.4 V) of the CV scan on the mixture of R⁴⁺ and D³⁺.

FIG. 52: Scheme 7 shows a graphic representation of Stage 4 (from −0.4 to +0.5 V) of the CV scan on the mixture of R⁴⁺ and D³⁺.

FIG. 53: Scheme 8 shows two possible pathways leading to the formation of the [D⊂R]*²⁺) bisradical complex, the key intermediate of the electron-catalysed molecular recognition process.

FIG. 54: Scheme 9 shows two possible pathways involving the transformation from the [D⊂R]^{+2(•+)} bisradical complex to the [D⊂R]^{+3(•+)} trisradical complex, the final product of the electron-catalysed molecular recognition process. SET, single-electron transfer.

### DETAILED DESCRIPTION OF THE INVENTION

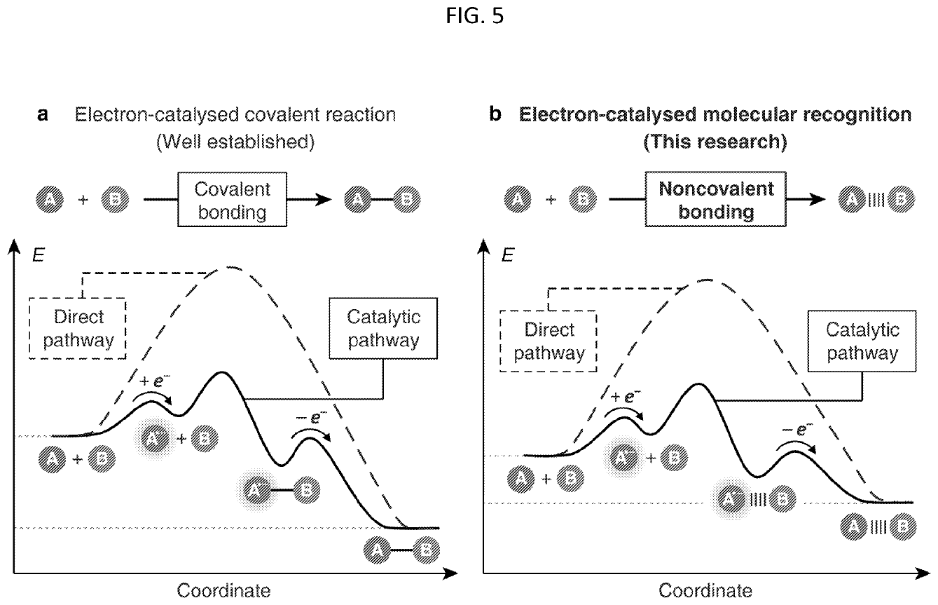

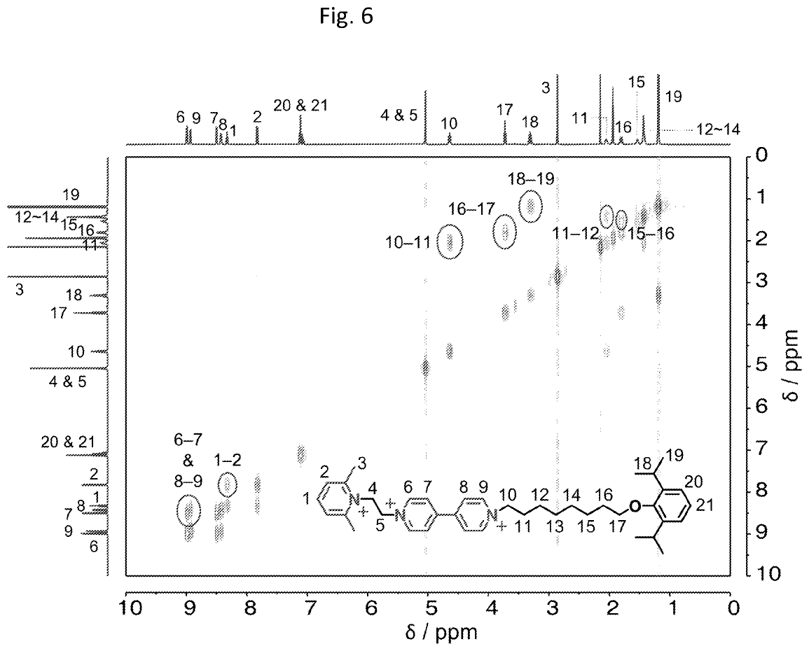

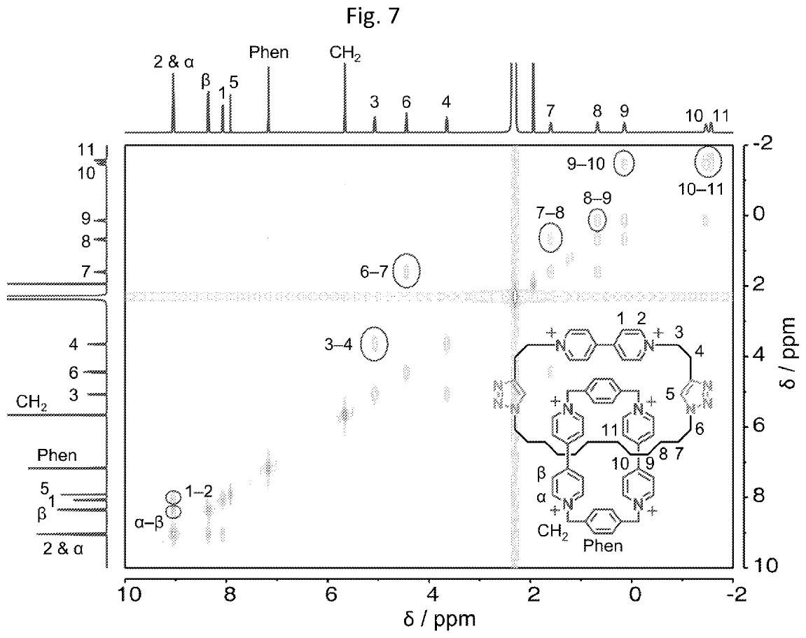

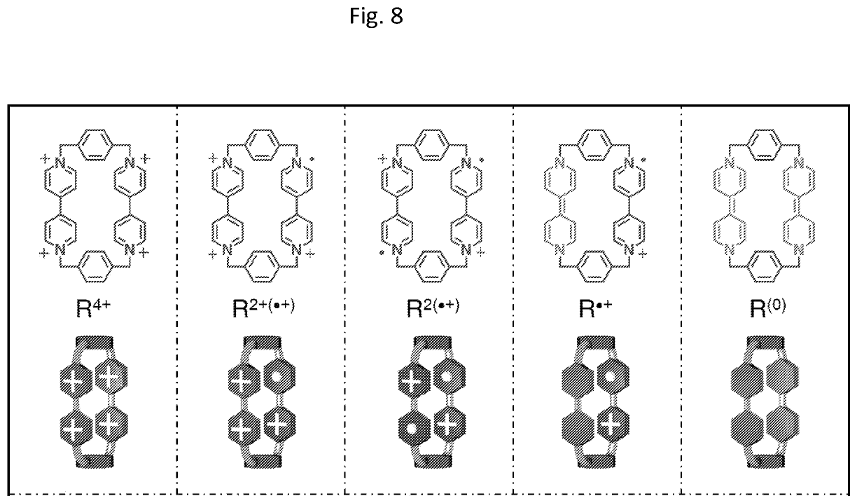

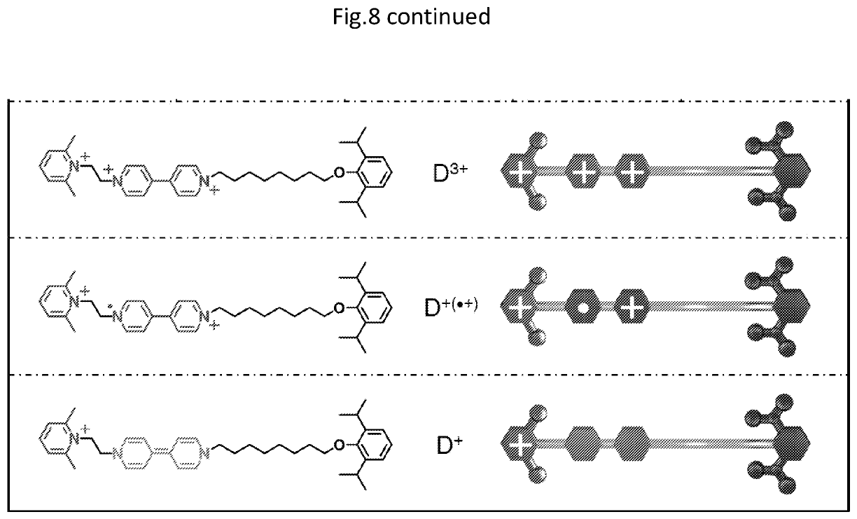

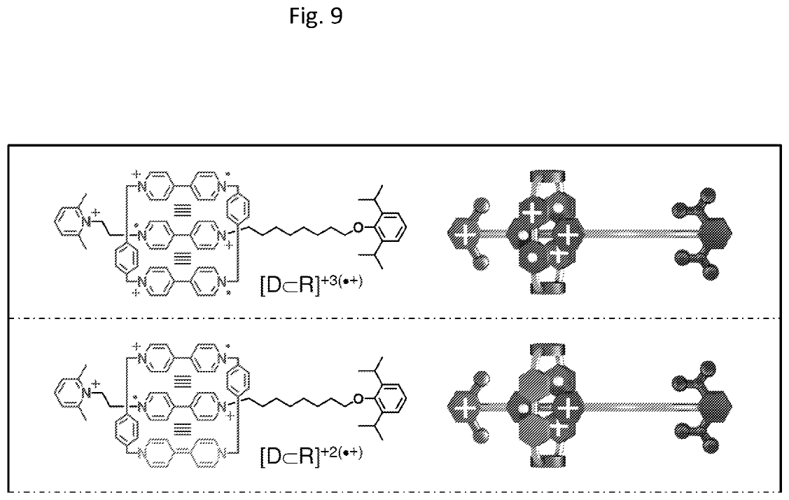

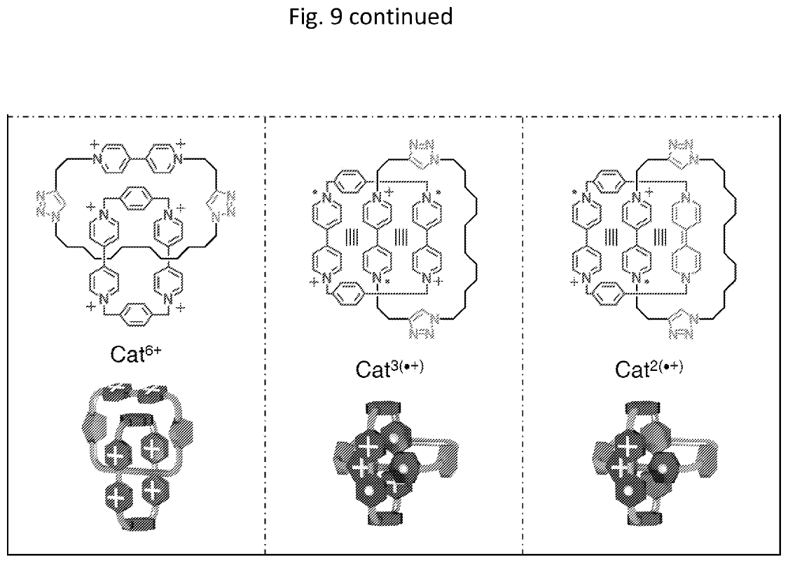

Disclosed herein is a versatile strategy to facilitate molecular recognition by electron catalysis¹⁷ in the realm of supramolecular noncovalent chemistry. As shown in the Examples, we show that the formation of a complex between substrates that a molecular recognition process is kinetically forbidden under ambient conditions can be accelerated dramatically upon the addition of catalytic amounts of a electrons from an electron source. It is therefore possible to control²³ the molecular recognition temporally and produce a nearly arbitrary distribution between substrates and complexes ranging from only substrates to equilibrium distributions. Such kinetically stable supramolecular systems²⁴ are difficult to obtain precisely by other means. The disclosed technology can be used to fine-tune noncovalent events, control assembly at different length scales²⁵⁻²⁷, and ultimately create new forms of complex matter²⁸⁻³⁰ Although the electron is an elementary particle, the electron can act^{17,18,31} as an effective catalyst by lowering energy barriers of molecular recognition (FIG. 5). The strategy of electron catalysis is applicable to molecular recognition processes with certain features: (i) at least one redox-active substrate capable of accepting (an) electron(s) rapidly, (ii) an energy barrier that can be decreased by the injection of electron(s), and (iii) a catalytic intermediate that is formed noncovalently from substrates and then transformed into the final product.

Molecular recognition refers to the specific interaction between two or more molecules through noncovalent bonding such as hydrogen bonding, metal coordination, hydrophobic forces, van der Waals forces, π-π interactions, halogen bonding, resonant interactions, and the like. In addition to these direct interactions, solvents can play an indirect role in driving molecular recognition in solution.

Electron catalyzed molecular recognition occurs by providing an electron to a multiplicity of substrates where at least one of the multiplicity of substrates is a redox-active substrate capable of accepting an electron from the electron source. By accepting the electron, the energy barrier for noncovalently forming a catalytic intermediate with a second substrate can be decreased. This allows for final products to be more easily accessed or for final products to be prepared that are kinetically inaccessible absence the substrate accepting the electron from the electron source.

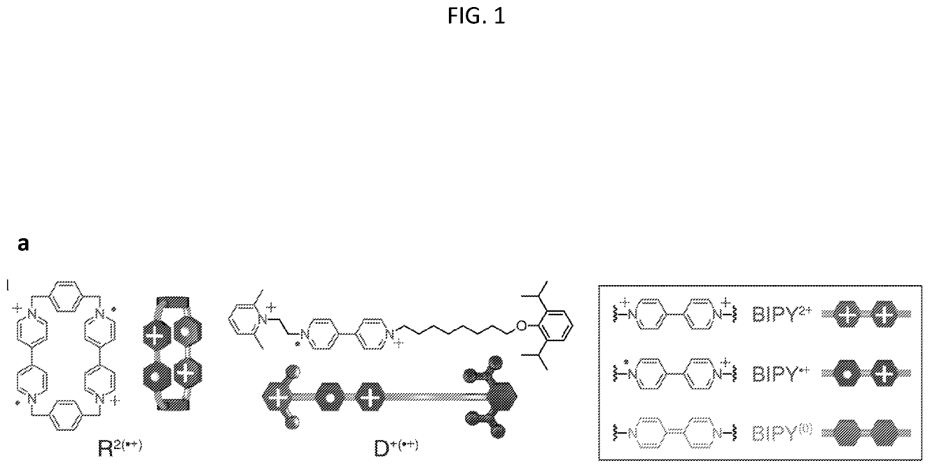

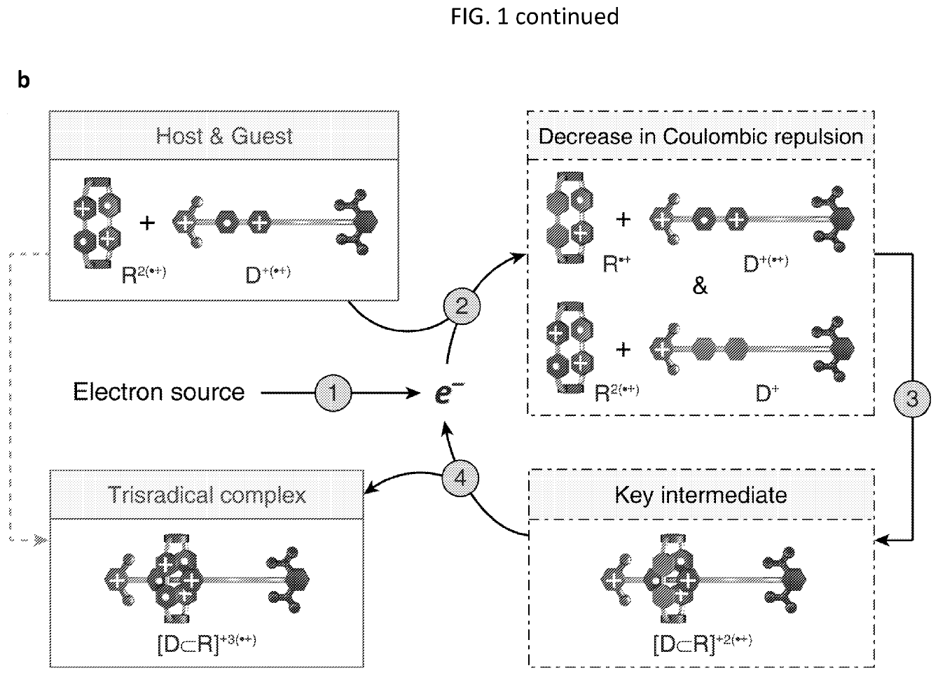

In some embodiments, the redox-active substrate comprises one or more bipyridinium (BIPY) units. BIPY units have three redox states, i.e., dicationic, radical cationic, and neutral states (FIG. 1). As a result, BIPY can be used to noncovalently form complexes by molecular recognition. For example, BIPY units may be useful for forming bisradical or trisradical complexes.

In some embodiments, the redox-active substrate is a macrocyclic ring. The macrocyclic ring may comprise one or more redox-active units, such as BIPY. In some embodiments, the macrocyclic ring comprises two separated redox-active units.

In some embodiments, the redox-active substrate is a dumbbell-shaped molecule. The dumbbell-shaped molecule may also comprises one or more redox-active units, such as BIPY.

Other redox-active substrates besides those that comprise BIPY may also be used with the disclosed technology. Exemplary substrates may include, without limitation, redox-active moieties such as nitroxides, ferrocene, phenothiazine and derivatives thereof, naphthalene diimides, perelene diimides, disulfide compounds, and diselenide compounds.

In some embodiments, the redox-active substrate is a macrocyclic ring and the second molecule is a dumbbell-shaped molecule. In other embodiments, the redox-active substrate is a dumbbell-shaped molecule and the second molecule is a macrocyclic ring. In either of these embodiments, the macrocyclic ring and the dumbbell-shaped molecular may be able to form a host-guest complex by molecular recognition where the macrocyclic ring hosts the dumbbell-shaped. As disclosed in the Examples, the barrier for forming a host-guest complex can be decreased by the substrate accepting an electron.

Other supramolecular systems besides host-guest systems may also be used with the disclosed technology. Exemplary supramolecular systems my include, without limitation, those based on hydrogen bonds, halogen bonds, hydrophobic effects, electrostatic interactions, metal-ligand coordination, π-π interactions, charge transfer interactions and so forth.

The electron source for providing the electron may be electrochemical or chemical. In some embodiments, the electron source is an electrochemical cell. As exemplified in the Examples, electrochemical cell may be undivided or divided. The working conditions, such as current intensity or stirring rate, can be modulated to control the rate of catalytic intermediate or final product formation.

In other embodiments, the electron source is a chemical initiator. As exemplified in the Examples, the chemical initiator can be a homogeneous or heterogeneous chemical initiator.

Exemplary homogeneous chemical initiators include, without limitation, a reduction product of the substrate (e.g., D⁺ and R⁽⁰⁾, the reduction products of R^{2(•+)} or D^{+(•+)}, respectively), a bisradical (e.g., 2]catenane Cat^{2(•+)}), or a reductant (e.g., bis(cyclopentadienyl) cobalt(II) (cobaltocene, CoCp₂), bis(pentamethylcyclopentadienyl) cobalt(II), Co(Cp*)₂ for short, and tetrakis(dimethylamino)ethylene (TDAE)). Exemplary heterogeneous chemical initiators include, without limitation, metals dusts (e.g., Mg, Al, Fe, Zn, or Cu dusts).

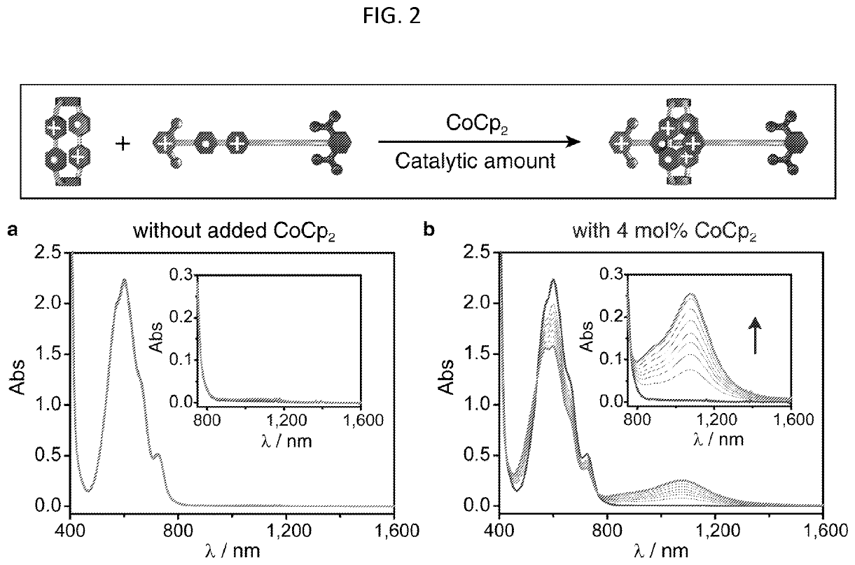

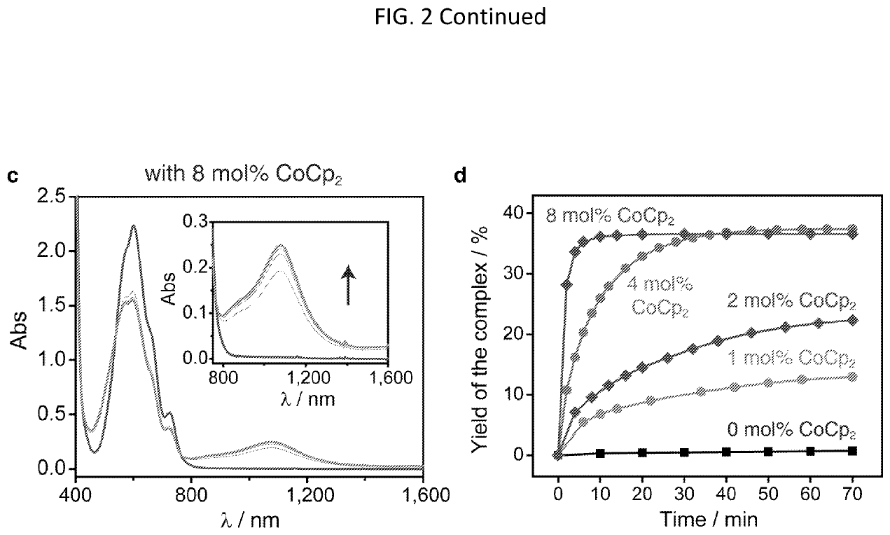

As demonstrated in the Examples, (FIG. 1a) an exemplary host-guest system is used to demonstrate electron-catalysed molecular recognition. In this system, the host is R^{2(•+)}, a ring bearing two separate bipyridinium radical cations (BIPY^{•+}). The dumbbell-shaped guest, D^{+(•+)}, comprises three parts—(i) a BIPY^{•+} unit in the middle, acting as the binding site for R^{2(•+)} forming a trisradical complex²² driven by radical-pairing interactions, (ii) a 2,6-diisopropylphenyl group on one end of the dumbbell-shaped molecule to prevent the threading of the ring by steric hindrance, and (iii) a 2,6-dimethylpyridinium (PY⁺) cation on the other end, which—in conjunction with the positively charged BIPY^{•+} unit-fulfils the role of a switchable energy barrier. Preliminary investigations (FIG. 2a) have indicated that the passage of R^{2(•+)} over the PY⁻ terminus of D^{+(•+)} is very slow on account of strong Coulombic repulsion. The energy barrier resulting from this repulsive interaction, which depends on the numbers of charges on the host and guest, can be adjusted (FIG. 1a) by changing the redox states³⁴ of bipyridinium units.

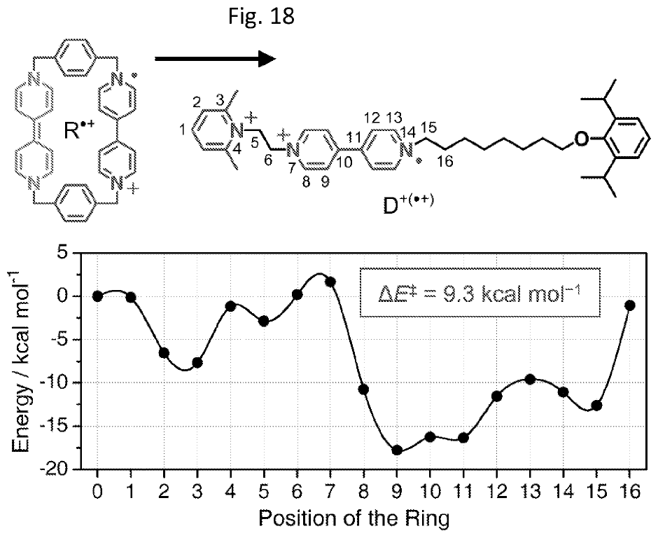

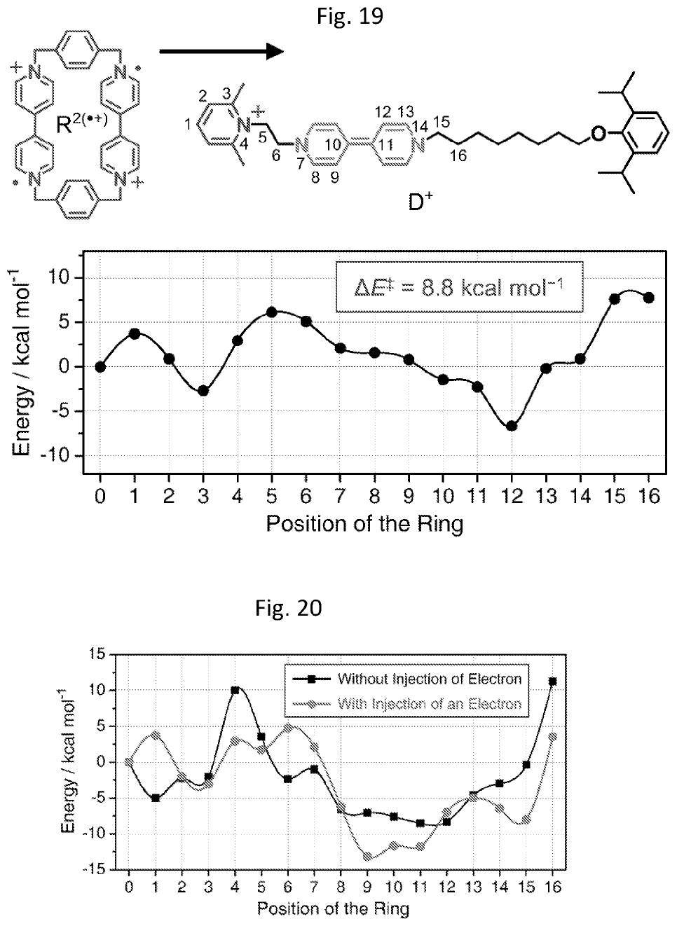

We propose (FIG. 1b) a design for electron-catalysed molecular recognition between R^{2(•+)} and D^{+(•+)} The catalysis is initiated by injection of an electron (Step 1), giving rise to the reduction of one BIPY^{•+} radical cation in the system to its neutral state—namely BIPY⁽⁰⁾. Either R^{2(•+)} or D^{+(•+)} can accept (Step 2) this electron to generate R^{•+} or D⁺, respectively. Since the electron is exchangeable, both the R^{•+}/D^{+(•+)} couple and the R^{2(•+)}/D⁺ couple exist in the system. The energy barriers associated with the passage of the ring over the PY⁺ terminus were determined by Quantum Mechanical calculations (FIGS. 18-19) to be 9.3 and 8.8 kcal mol⁻¹, respectively. Both values are lower than the energy barrier (15.0 kcal mol⁻¹, FIG. 17) associated with the direct binding between R^{2(•+)} and D^{+(•+)} without the additional electron. This result can be rationalized based on the diminished Coulombic repulsion between the two molecules. The decrease in the energy barrier contributes to the rapid formation (Step 3) of a [D⊂R]^{+2(•+)} bisradical complex, driven by a combination of radical-pairing and donor-acceptor interactions. This catalytic intermediate can be transformed to the final product, i.e., the [D⊂R]^{3+(•+)} trisradical complex, concomitantly releasing (Step 4) an electron to R^{2(•+)} or D^{+(•+)} to close the catalytic cycle. We anticipate that catalytic amounts of electrons can induce a dramatic acceleration of the molecular recognition between R^{2(•+)} and D^{+(•+)}, even although this host-guest complexation is all but kinetically forbidden under ambient conditions.

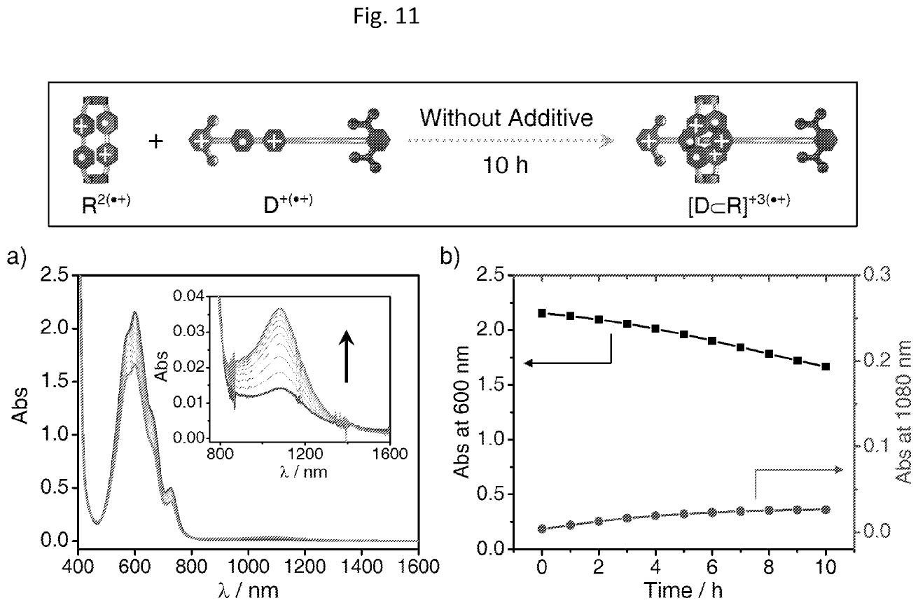

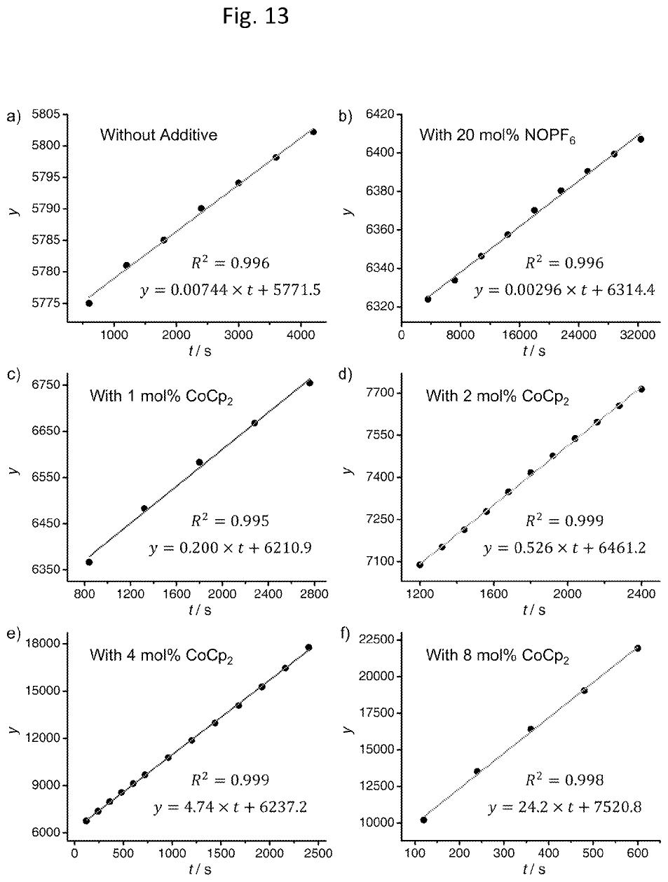

To test the efficacy of this electron catalysis approach, molecular recognition between R^{2(•+)} and D^{+(•+)} was carried out in acetonitrile (MeCN) solution and monitored by UV/Vis/near-infrared (NIR) spectroscopy. The combination of only R^{2(•+)} and D^{+(•+)} brings about (FIG. 2a and FIG. 11) little change in the absorption spectrum for at least 10 h, indicating that there is negligible formation of the [D⊂R]^{+3 (•+)} trisradical complex. Upon addition of 4 mol % cobaltocene-CoCp₂, which is capable of transforming³⁴ BIPY^{•+} to BIPY⁽⁰⁾—we observed (FIG. 2b) the appearance of a NIR absorption band (λₘₐₓ=1080 nm) characteristic²² of the trisradical complex, accompanied by the decay in the absorption (λₘₐₓ=600 nm) for the uncomplexed BIPY^{•+}. This spectral change, which signifies the onset of molecular recognition, reaches a constant value in 70 min. Fitting (FIG. 13) the data with a kinetic model for a second-order reversible reaction, the rate constant of this supramolecular process in the presence of 4 mol % CoCp₂ was determined to be 4.74 L mol⁻¹ s⁻¹, showing a 640-fold acceleration compared to the rate constant (7.44×10⁻³ L mol⁻¹ s⁻¹) observed in the absence of CoCp₂.

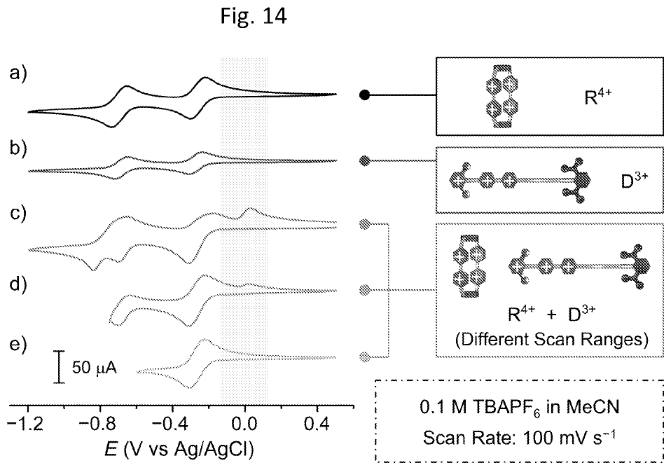

Increasing the amount of CoCp₂ from 4 to 8 mol % expedites (FIG. 2c) the molecular recognition process, rendering it complete in 10 min with a similar yield of the [D⊂R]^{+3(•+)} trisradical complex. Decreasing (FIG. 2d) the amount of CoCp₂ from 4 to 2 and 1 mol %, this process is still promoted albeit at slower rates. Comparison (FIG. 2d) between the kinetic traces in the presence of 4 and 8 mol % CoCp₂ reveals the catalytic nature of the process: a small amount of CoCp₂ can accelerate the kinetics but has little influence on the thermodynamic equilibrium. Cyclic voltammetry (CV) measurements (FIG. 14) confirm that the injection of electrons promotes the molecular recognition process, in which the reduction from BIPY^{•+} to BIPY⁽⁰⁾, along with the reoxidation from BIPY⁽⁰⁾ back to BIPY^{•+}, proves to be indispensable for the formation of the [D⊂R]^{+3(•+)} trisradical complex on the time scale of the CV experiments.

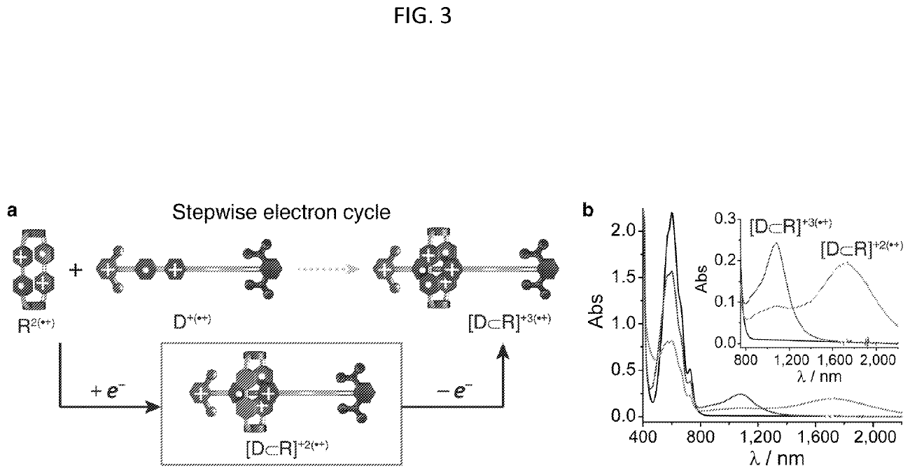

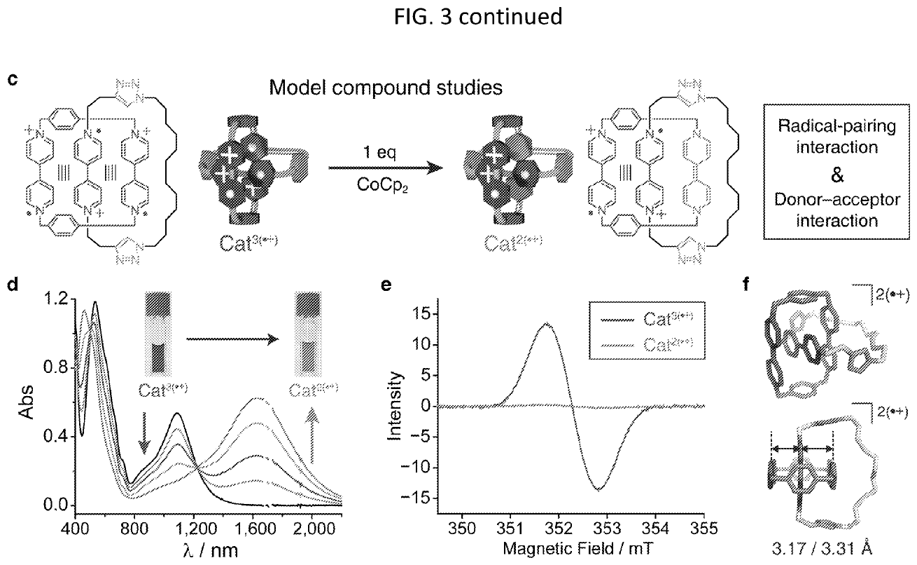

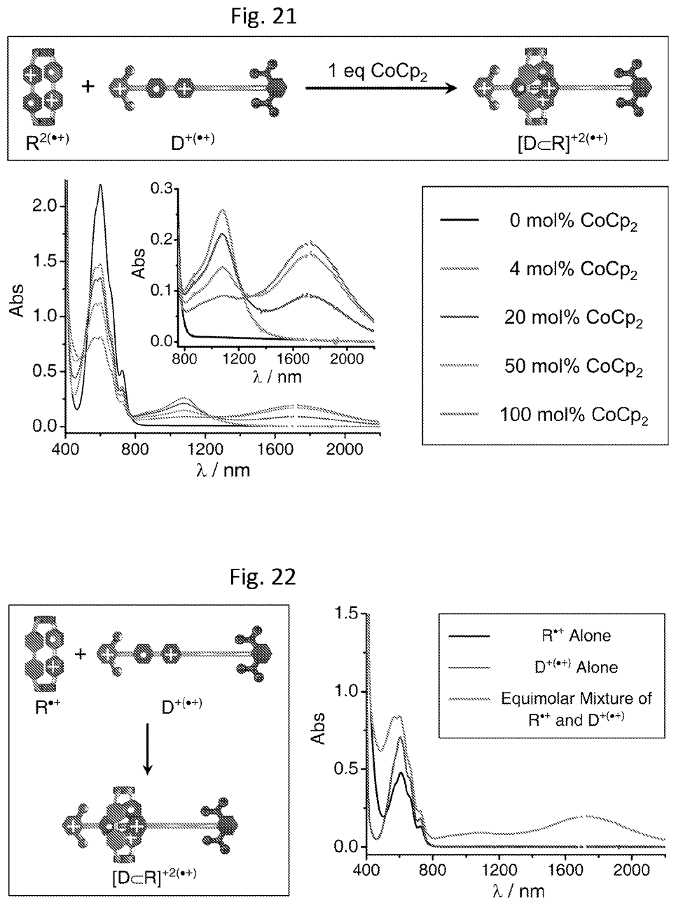

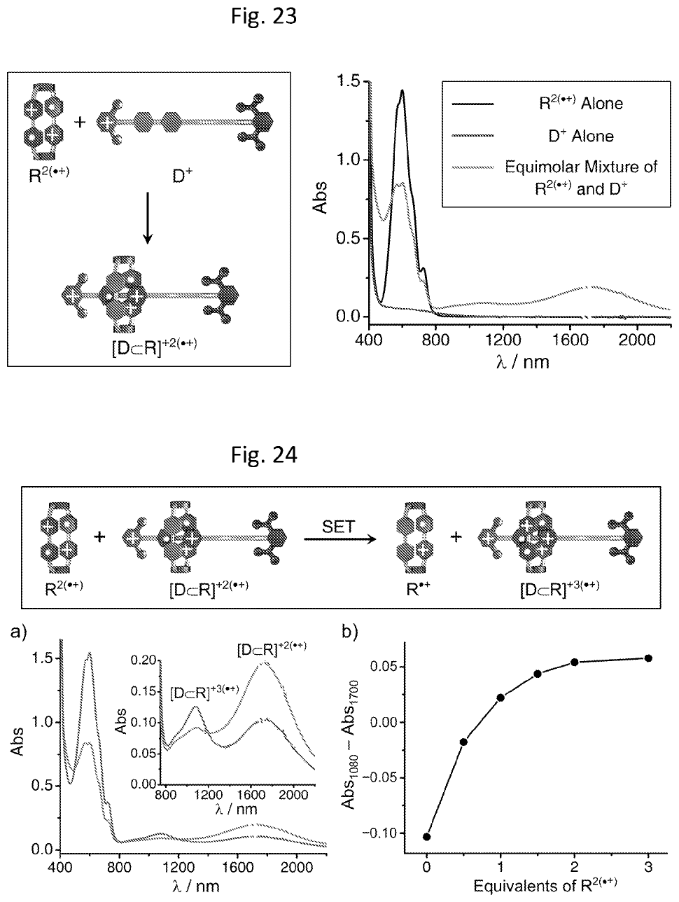

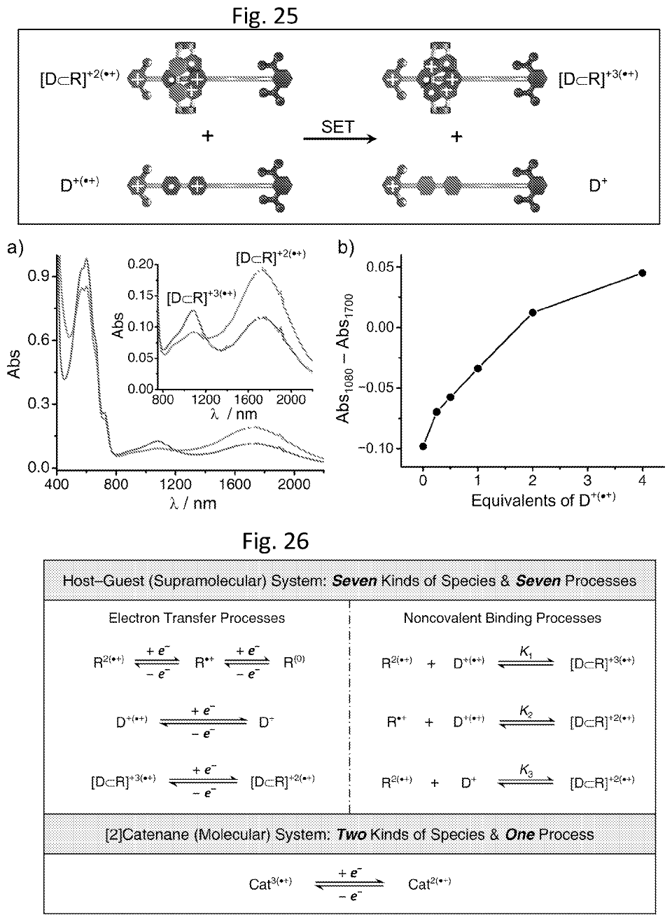

To elucidate the mechanism of this molecular recognition, we performed (FIG. 3a) electron catalysis in a stepwise manner. In the first step, 1 molar equivalent of CoCp₂ was added to the equimolar mixture of R^{2(•+)} and D^{+(•+)}, leading to the instantaneous appearance (FIG. 3b, [D⊂R]^{+2(•+)}, and FIGS. 21-23) of a new, broad NIR absorption band at λₘₐₓ=1700 nm. This absorption band, different from the well-recognized absorption (λₘₐₓ=1080 nm) for the trisradical complex, was ascribed to the [D⊂R]^{+2(•+)} bisradical complex. In the second step, further addition of excess of D^{+(•+)} or R^{2(•+)} into the system results in the transformation from the [D⊂R]^{+2(•+)} bisradical complex to the [D⊂R]^{+3(•+)} trisradical complex, as indicated by the absorption peak shifting (FIG. 3b, [D⊂R]^{+3(•+)}, and FIGS. 24-25) from 1700 to 1080 nm. We suggest that this bisradical complex serves as the key intermediate in the electron catalysis.

Further investigation of this intermediate was hindered because the combination of R^{2(•+)} D^{+(•+)} and CoCp₂ constitutes a complicated supramolecular system (FIG. 26), involving multiple, interrelated host-guest complexation and electron transfer processes. A well-defined molecular system is therefore necessary for unravelling the structure and properties of the bisradical complex. A [2]catenane (Cat⁶⁺)—wherein a R⁴⁺ ring bearing two BIPY²⁺ units and another ring containing one BIPY²⁺ unit are mechanically interlocked—was synthesized³⁵ and employed as a model compound. Cat⁶⁺ was first of all reduced to its trisradical tricationic state, Cat^{3(•+)}, followed by titration (FIG. 3c) with 1 molar equivalent of CoCp₂. During the titration, the colour of solution changed from purple to brown. Correspondingly, the characteristic absorption for the trisradical tricationic state (λₘₐₓ=1080 nm) was found to decrease gradually, accompanied by the elevation (FIG. 3d) of a new, red-shifted absorption band (λₘₐₓ=1640 nm) with isobestic points at 506 and 1219 nm. These observations indicate the quantitative transformation from Cat^{3(•+)} to Cat^{2(•+)}. Notably, the absorption of Cat^{2(•+)} is close to that of the [D⊂R]^{+2(•+)} bisradical complex, suggesting the structural similarity of their chromophores. The electron paramagnetic resonance (EPR) signal (FIG. 3e) of Cat^{2(•+)} is nearly silent, supporting its diamagnetic nature in contrast with the paramagnetic nature of Cat^{3(•+)}.

Single-crystal X-ray diffraction analysis (FIG. 3f) verified the intercomponent binding within Cat^{2(•+)}, in which the R ring encircles the BIPY unit belonging to the other ring. The centroid-to-centroid distances between adjacent BIPY units in Cat^{2(•+)} were found to be 3.17 and 3.31 Å, indicating³⁶ the existence of (i) a radical-pairing interaction between two adjacent BIPY^{•+} radical cations and (ii) a donor-acceptor interaction between an electron-deficient BIPY^{•+} radical cation and an electron-rich BIPY⁽⁰⁾ neutral unit. These interactions present in Cat^{2(•+)} are also responsible for the formation of its non-interlocked counterpart, i.e., the [D⊂R]^{+2(•+)} bisradical complex. Through the analysis of the model compound, we have identified the bisradical complex as the key intermediate during the electron-catalysed molecular recognition process, supporting strongly the mechanism proposed in FIG. 1b.

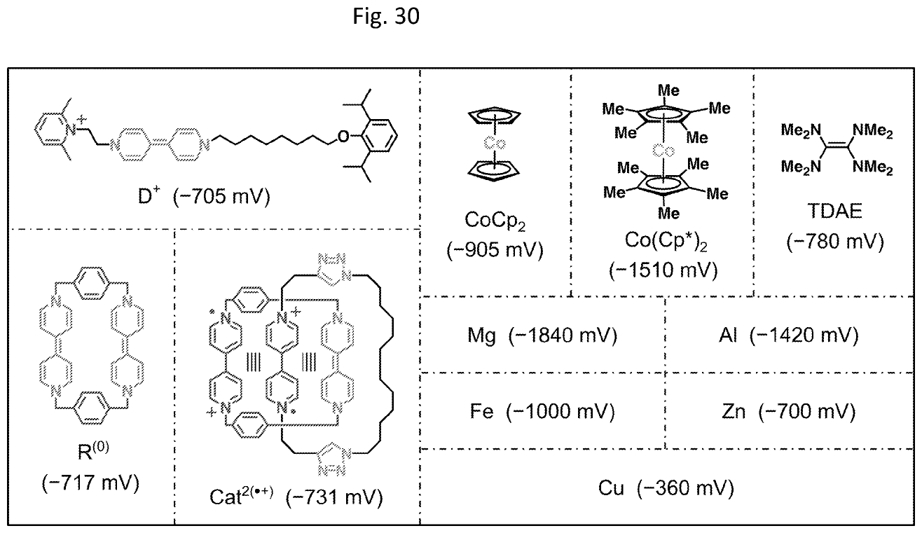

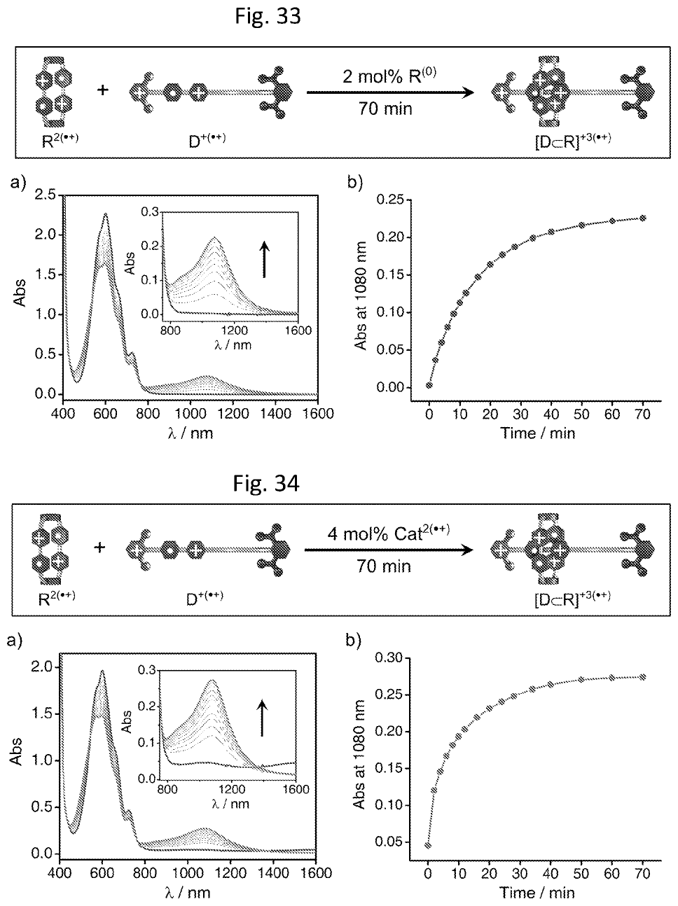

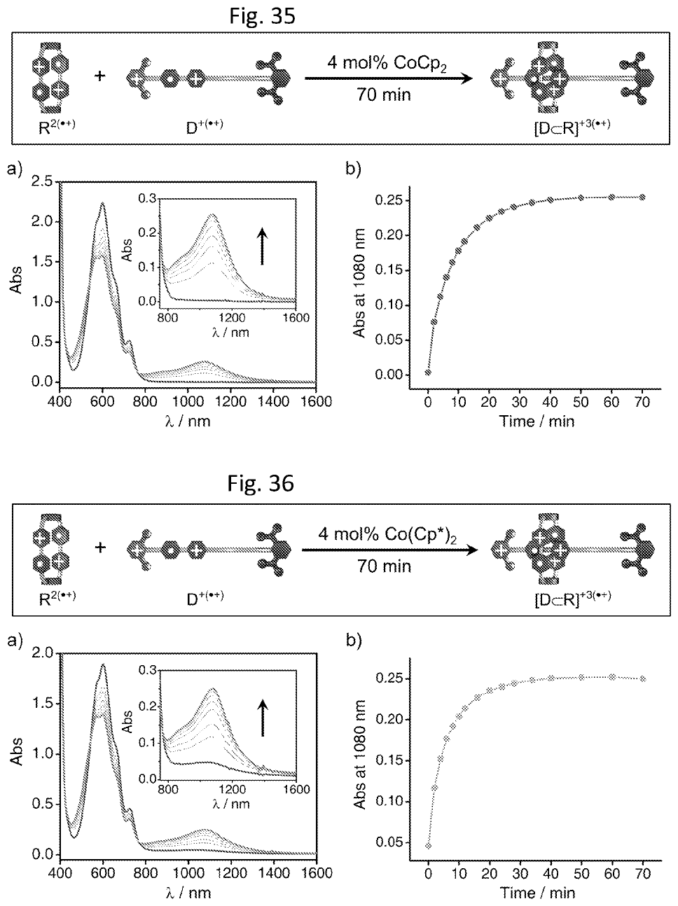

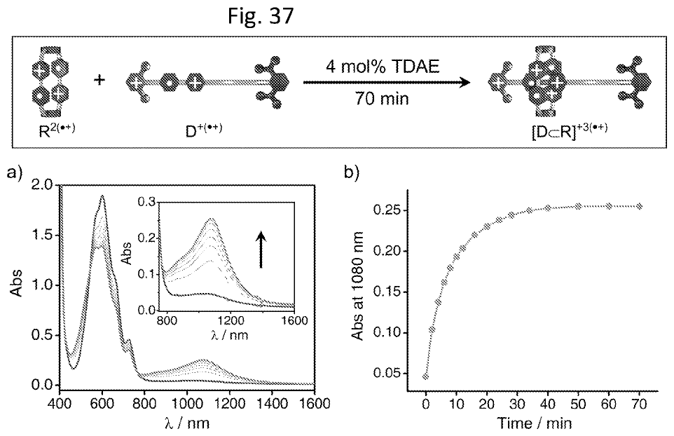

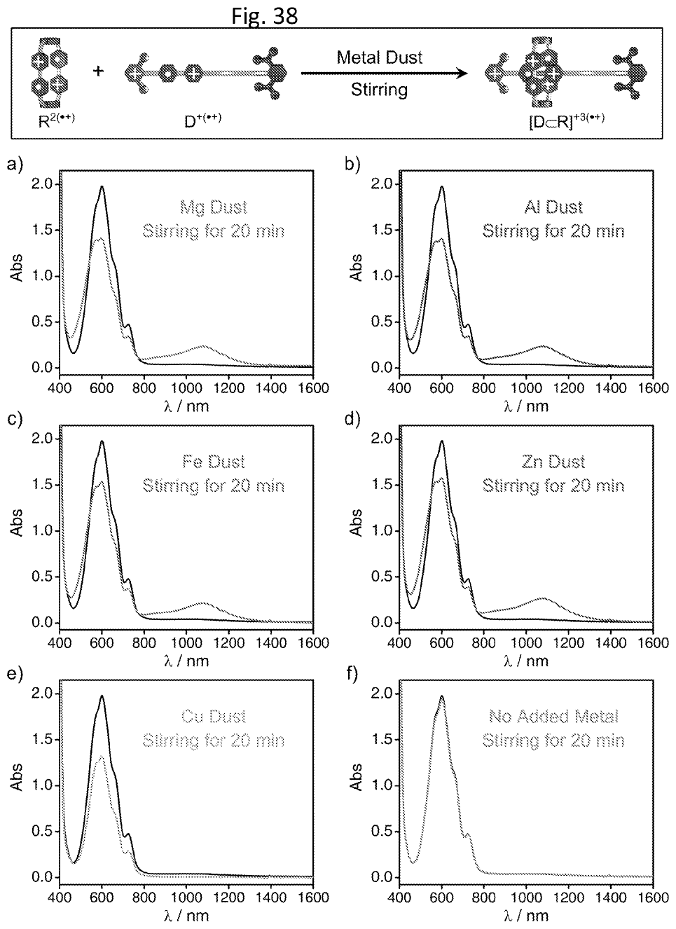

The electron is the catalyst for the molecular recognition between R^{2(•+)} and D^{+(•+)}, while CoCp₂ serves only as a chemical electron source and an initiator of the process. A corollary¹⁷ to this mechanistic insight is that any chemical reagents with (i) appropriate reduction potentials to transform BIPY^{•+} into BIPY⁽⁰⁾ and (ii) reasonable electron-transfer rates can, in principle, initiate the electron catalysis. Hence, we have screened a variety of chemical reagents, including active metals, metal complexes and organic reductants, and demonstrated their effectiveness (FIGS. 30-38) in promoting molecular recognition. This finding indicates that the strategy of electron catalysis is less dependent on the structures of chemical initiators, an observation showing our approach to be fundamentally different from previous strategies¹³⁻¹⁵ that rely on elaborately designed catalysts.

The mechanistic understanding of electron catalysis prompted us to control the molecular recognition by electrochemical means a clean and straightforward way to inject electrons. When we perform electrolysis on the mixture of R^{2(•+)} and D^{+(•+)} in an undivided cell, the simultaneous cathodic reduction and anodic oxidation of BIPY^{•+} radical cations are expected to occur, generating equal amounts of BIPY⁽⁰⁾ and BIPY²⁺, respectively. These changes in the redox state are transient, because a spontaneous, mass transport-controlled single-electron transfer (SET) between BIPY⁽⁰⁾ and BIPY²⁺ will restore BIPY^{•+}. We surmise that if the lifetime of BIPY⁽⁰⁾ and BIPY²⁺ is sufficiently longer than the time required for molecular recognition, the injection and removal of electrons at electrodes will be able to promote the complexation between R^{2(•+)} and D^{+(•+)} in solution.

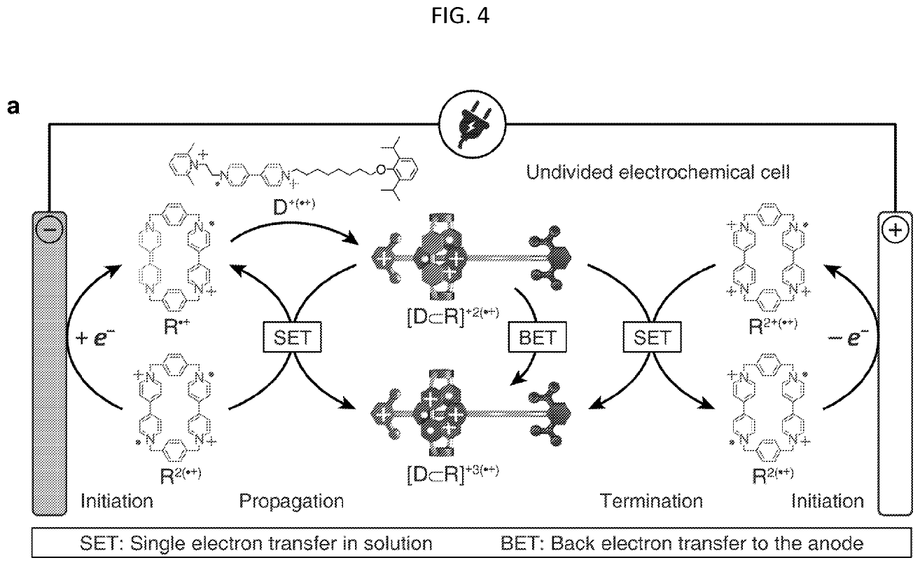

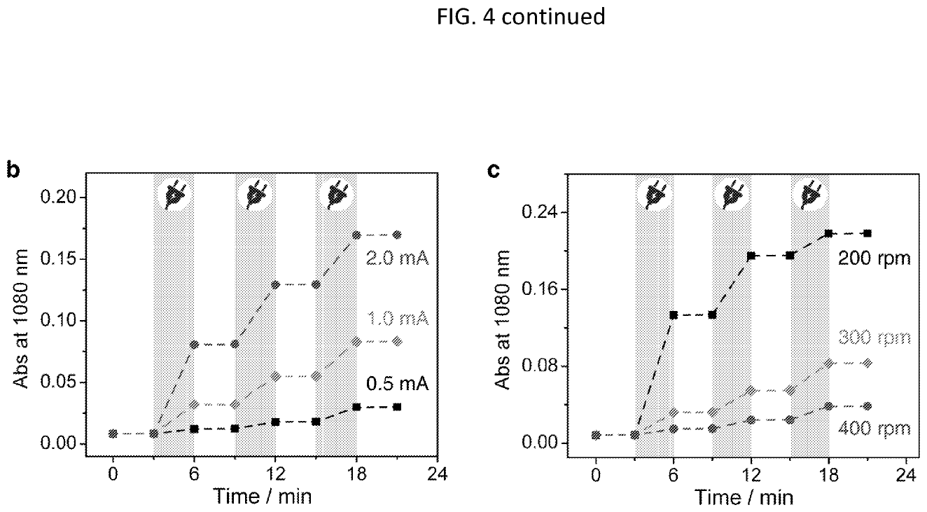

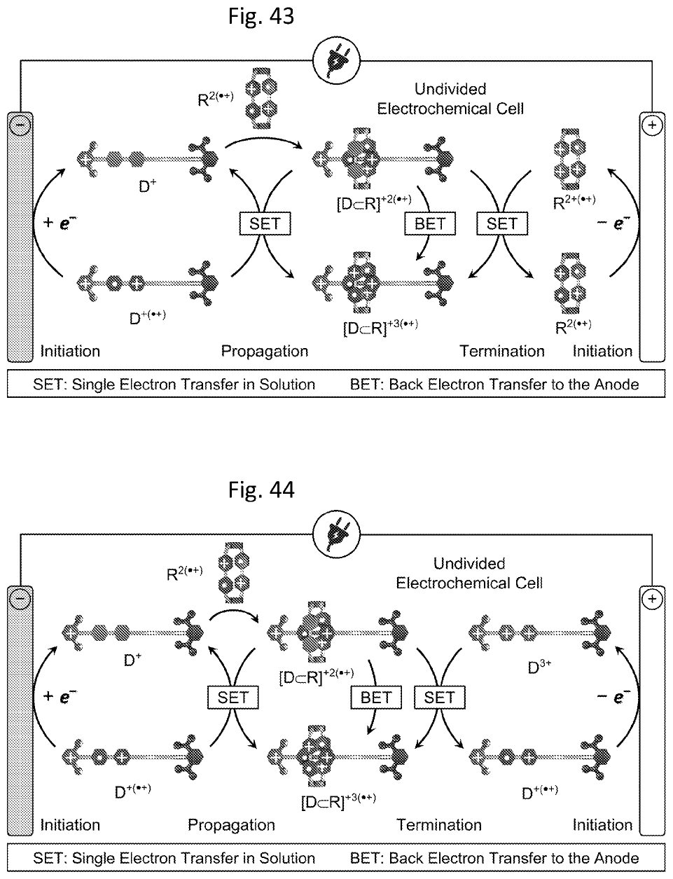

Since both R^{2(•+)} and D^{+(•+)} contain BIPY^{•+} radical cations that can accept/lose electrons from/to the electrode, there are several possible pathways for molecular recognition to occur during the electrochemical process. FIG. 4a illustrates one of the possibilities in which the cathodic reduction and anodic oxidation are both imposed on R^{2(•+)}, generating equal amounts of R^{•+} and R^{2+(•+)}, respectively. After the electrochemical initiation, some of the R can bind rapidly with D^{+(•+)}, leading to the formation of the [D⊂R]^{+2(•+)} bisradical complex. Subsequently, this intermediate can undergo SET with R^{2(•+)}, affording a [D⊂R]^{+3(•+)} trisradical complex as the final product and generating a new R^{•+} to restart the cycle (propagation). The catalytic cycle is terminated when the [D⊂R]^{+2(•+)} bisradical complex undergoes SET with R^{2+(•+)} to recover R^{2(•+)} as the starting material or transfers an electron back to the anode and generates a [D⊂R]^{+3(•+)} trisradical complex. Several other possible pathways, following similar logic, are illustrated in FIGS. 42-44. During the electrolysis under stirring, the number of catalytic cycles prior to termination depends on the convection rates of the intermediates. Detailed discussions regarding theoretical analyses of electrochemically controlled molecular recognition can be found in the Examples.

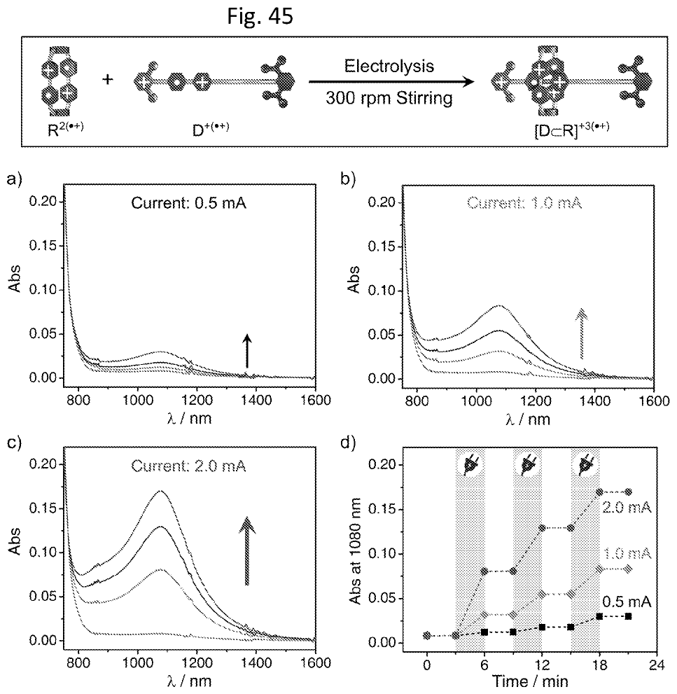

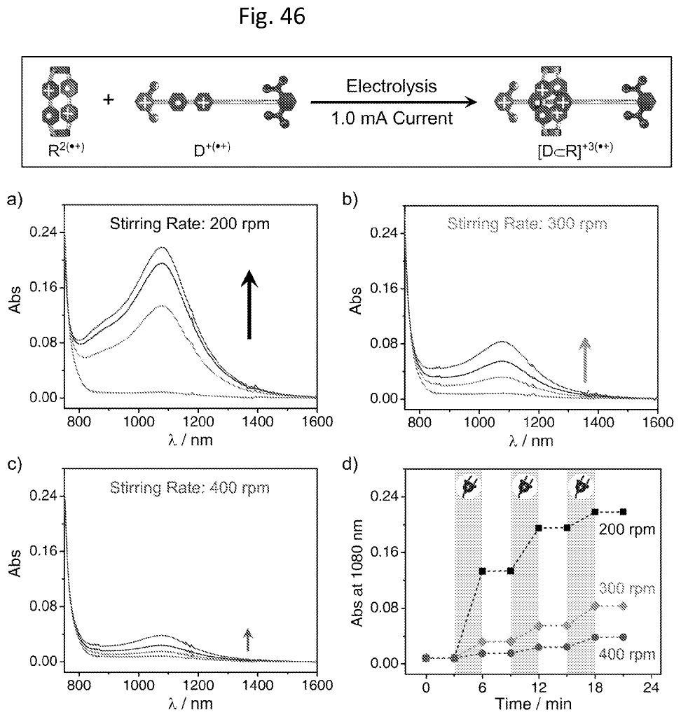

In order to validate the proposed electrochemical mechanism, the electrolysis of a MeCN solution containing R^{2(•+)} and D^{+(•+)} was conducted at a constant current and stirring rate under a N₂ atmosphere, followed by sampling the solution periodically to record the UV/Vis/NIR spectrum. After the first 3 min of electrolysis, we observed (FIG. 4b, 4c and FIGS. 45-46) an increase in the absorption band characteristic of the [D⊂R]^{+3(•+)} trisradical complex. The measurement of the same sample—after being maintained in the spectrometer for 3 min—revealed that the absorbance underwent no change. Repeated on/off cycling of electricity results in the intermittent increase in the formation of trisradical complexes. These observations have demonstrated²³ a rare example of temporally controlled supramolecular processes, allowing us to produce kinetically stable systems²⁴ with well-defined distributions between substrates and complexes, i.e., by applying electricity for a pre-determined time, we can create a system with any molar ratio between substrates and complexes ranging from zero to the equilibrium value. In contrast with the chemical addition of electrons that cannot be removed simultaneously, the electrolysis in an undivided cell allows molecular recognition to be controlled temporally on account of the constant delivery of anodically formed oxidants that curb the propagation of catalytic cycles and quench the molecular recognition after switching off the electricity.

In addition to the temporal control, the kinetics of molecular recognition is tuneable by adjusting the working conditions of electrolysis, such as current intensity and stirring rate. On the one hand, increasing the current from 0.5 to 1.0 to 2.0 mA—with the same stirring rate at 300 rpm—gives rise to an increased speed (FIG. 4b) in molecular recognition. On the other hand, comparison (FIG. 4c) between the kinetic traces at 200, 300 and 400 rpm stirring rate—with fixed current at 1.0 mA-reveals that molecular recognition is slowed down when stirring the solution more vigorously. A plausible reason for this observation is that in an undivided cell, faster convection will expedite SET between BIPY⁽⁰⁾ and BIPY²⁺ and thus decrease the lifetime of both species, given the consideration that the BIPY⁽⁰⁾ unit is responsible for facilitating molecular recognition. In short, extending the paradigm of electron catalysis from synthetic covalent chemistry to supramolecular noncovalent chemistry, unleashes the tremendous power of the electron in promoting and controlling self-assembly processes.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1 Lehn, J.-M. Supramolecular chemistry—Scope and perspectives molecules, supermolecules, and molecular devices (Nobel lecture). Angew. Chem. Int. Ed Engl. 27, 89-112 (1988).
2 Cram, D J. The design of molecular hosts, guests, and their complexes (Nobel lecture). Angew. Chem. Int. Ed. Engl. 27, 1009-1020 (1988).
3 Philp, D. & Stoddart, J. F. Self-assembly in natural and unnatural systems. Angew. Chem. Int. Ed. Engl. 35, 1154-1196 (1996).
4 Persch, E., Dumele, O. & Diederich, F. Molecular recognition in chemical and biological systems. Angew. Chem. Int. Ed. 54, 3290-3327 (2015).
5 Aida, T., Meijer, E. W. & Stupp, S. I. Functional supramolecular polymers. Science 335, 813-817 (2012).
6 Das, K., Gabrielli, L. & Prins, L. J. Chemically fueled self-assembly in biology and chemistry. Angew. Chem. Int. Ed. 60, 20120-20143 (2021).
7 Weiβenfels, M., Gemen, J. & Klajn, R. Dissipative self-assembly: Fueling with chemicals versus light. Chem 7, 23-37 (2021).
8 Yin, Z. et al. Dissipative supramolecular polymerization powered by light. CCS Chem. 1, 335-342 (2019).
9 Wiester, M. J., Ulmann, P. A. & Mirkin, C. A. Enzyme mimics based upon supramolecular coordination chemistry. Angew. Chem. Int. Ed 50, 114-137 (2010).
10 Webber, M. J., Appel, E. A., Meijer, E. W. & Langer, R. Supramolecular biomaterials. Nat. Mater. 15, 13-26 (2015).
11 Amabilino, D. B., Smith, D. K. & Steed, J. W. Supramolecular materials. Chem. Soc. Rev. 46, 2404-2420 (2017).
12 Wang, Y. et al. What molecular assembly can learn from catalytic chemistry. Chem. Soc. Rev. 43, 399-411 (2014).
13 Turberfield, A. J. et al. DNA fuel for free-running nanomachines. Phys. Rev. Lett. 90, 118102 (2003).
14 Zhang, D. Y., Turberfield, A. J., Yurke, B. & Winfree, E. Engineering entropy-driven reactions and networks catalyzed by DNA. Science 318, 1121-1125 (2007).
15 Song, T. & Liang, H. Synchronized assembly of gold nanoparticles driven by a dynamic DNA-fueled molecular machine. J Am. Chem. Soc. 134, 10803-10806 (2012).
16 Li, H. et al. Proton-assisted self-assemblies of linear di-pyridyl polyaromatic molecules at solid/liquid interface. J. Phys. Chem. C 116, 21753-21761 (2012).
17 Studer, A. & Curran, D. P. The electron is a catalyst. Nat. Chem. 6, 765-773 (2014).
18 Francke, R. & Little, R. D. Electrons and holes as catalysts in organic electrosynthesis. ChemElectroChem 6, 4373-4382 (2019).
19 Yan, M., Kawamata, Y. & Baran, P. S. Synthetic organic electrochemical methods since 2000: On the verge of a renaissance. Chem. Rev. 117, 13230-13319 (2017).
20 Prier, C. K., Rankic, D. A. & MacMillan, D. W. C. Visible light photoredox catalysis with transition metal complexes: Applications in organic synthesis. Chem. Rev. 113, 5322-5363 (2013).
21 Romero, N. A. & Nicewicz, D. A. Organic photoredox catalysis. Chem. Rev. 116, 10075-10166 (2016).
22 Trabolsi, A. et al. Radically enhanced molecular recognition. Nat. Chem. 2, 42-49 (2010).
23 Aubert, S., Bezagu, M., Spivey, A. C. & Arseniyadis, S. Spatial and temporal control of chemical processes. Nat. Rev. Chem. 3, 706-722 (2019).
24 Mattia, E. & Otto, S. Supramolecular systems chemistry. Nat. Nanotechnol. 10, 111-119 (2015).
25 Whitesides, G. M. Self-assembly at all scales. Science 295, 2418-2421 (2002).
26 Harada, A., Kobayashi, R., Takashima, Y., Hashidzume, A. & Yamaguchi, H. Macroscopic self-assembly through molecular recognition. Nat. Chem. 3, 34-37 (2010).
27 Santos, P. J., Gabrys, P. A., Zornberg, L. Z., Lee, M. S. & Macfarlane, R. J. Macroscopic materials assembled from nanoparticle superlattices. Nature 591, 586-591 (2021).
28 Lehn, J. M. Toward self-organization and complex matter. Science 295, 2400-2403 (2002).
29 Vantomme, G. & Meijer, E. W. The construction of supramolecular systems. Science 363, 1396-1397 (2019).
30 Pezzato, C., Cheng, C., Stoddart, J. F. & Astumian, R. D. Mastering the non-equilibrium assembly and operation of molecular machines. Chem. Soc. Rev. 46, 5491-5507 (2017).
31 Luca, O. R., Gustafson, J. L., Maddox, S. M., Fenwick, A. Q. & Smith, D. C. Catalysis by electrons and holes: Formal potential scales and preparative organic electrochemistry. Org. Chem. Front. 2, 823-848 (2015).
32 Whitesides, G. M. et al. Noncovalent synthesis: Using physical-organic chemistry to make aggregates. Acc. Chem. Res. 28, 37-44 (1995).
33 Reinhoudt, D. N. Synthesis beyond the molecule. Science 295, 2403-2407 (2002).
34 Frasconi, M. et al. Redox control of the binding modes of an organic receptor. J. Am. Chem. Soc. 137, 11057-11068 (2015).
35 Wang, Y. et al. Symbiotic control in mechanical bond formation. Angew. Chem. Int. Ed. 55, 12387-12392 (2016).
36 Cai, K. et al. Molecular Russian dolls. Nat. Commun. 9, 5275 (2018).

### Examples

### General Methods for Conducting Chemically Initiated Molecular Recognition.

Homogenous chemical initiators. A screw-cap cuvette was charged with MeCN solutions of R^{2(•+)} (0.3 mM, 500 μL, 1 equiv) and D^{+(•+)} (0.3 mM, 500 μL, 1 equiv) in a N₂-filled glovebox. A specific amount of chemical initiator—for example, a MeCN solution of CoCp₂ (1.5 mM, 4 μL, 4 mol %)—was added to the system. After rapid mixing of the solution, the sealed cuvette was transferred to a spectrometer and the UV/Vis/NIR spectra were recorded in 2 min intervals. The kinetic trace of this process was obtained by plotting the absorbance at 1080 nm against time.

Heterogenous chemical initiators. MeCN solutions of R^{2(•+)} (0.15 mM, 500 μL, 1 equiv) and D^{+(•+)} (0.15 mM, 500 μL, 1 equiv) were combined in a vial, followed by the addition of excess of metal (e.g., Zn) dust. The suspension was stirred in a N₂-filled glovebox for 20 min. After removing the solid by filtration, the resulting solution was transferred to a screw-cap cuvette to record the UV/Vis/NIR spectrum.

### General Methods for Conducting Electrochemically Controlled Molecular Recognition.

Electrochemically controlled molecular recognition between R^{2(•+)} and D^{+(•+)} was conducted in the N₂-filled glovebox using an IKA© Electrasyn 2.0 Device. The set-up was an undivided electrochemical cell, in which both the cathode and anode are reticular vitreous carbon electrodes. In a typical procedure, a MeCN solution (9 mL) containing R^{2(•+)} (0.15 mM), D^{+(•+)} (0.15 mM) and TBAPF₆ (0.05 μM) was electrolysed for 3 min at a constant current (e.g., 1.0 mA) and stirring rate (e.g., 300 rpm). Subsequently, the current was switched off and the solution was allowed to stand for 3 min. The overall process consisted of three on/off cycles. The molecular recognition during the intermittent electrolysis was monitored by sampling the solution in 3 min interval and recording its UV/Vis/NIR spectrum.

### 1. Materials and General Methods

All reagents were purchased from commercial supplies (Sigma-Aldrich, TCI or Fisher) and were used as received. Cyclobis(paraquat-p-phenylene) tetrakis(hexafluorophosphate) (R•4PF₆) and several synthetic building blocks (S1•2PF₆, S2, S3•2PF₆ and S4) were synthesized according to the previously reported procedures¹⁻⁴. Thin layer chromatography (TLC) was performed on silica gel 60 F254 (E Merck). Column chromatography, including normal phase (RediSep Rf Gold® Normal-Phase Silica) and reversed-phase (RediSep Rf Gold® Reversed-Phase C18) methods, were carried out using CombiFlash® Automation Systems (Teledyne ISCO). Nuclear magnetic resonance (NMR) spectra were recorded at room temperature on a Bruker Avance III 500 MHz spectrometer equipped with DCH CryoProbe, with working frequencies of 500 MHz for ¹H and 126 MHz for ¹³C nuclei, respectively. Chemical shifts are reported in ppm relative to the signals corresponding to the residual non-deuterated solvents (CD₃CN: δH=1.94 ppm and δC=118.26 ppm). The ¹³C NMR spectra were recorded with the simultaneous decoupling of proton nuclei. High-resolution mass spectra were recorded on an Agilent 6210 Time-of-Flight (TOF) LC-MS coupled with an electrospray ionization (ESI) source. The samples were introduced into the ESI probe using direct infusion with a flow rate of 0.6 mL min⁻¹. UV/Vis/Near-infrared (NIR) spectra were recorded at room temperature on a Shimadzu UV-3600 spectrophotometer. All the samples were prepared freshly (from stock solutions or from solids) in a N₂-filled glovebox and then transferred into a sealed cuvette for recording the spectra. Path length was set to 4 mm. The sharp absorption peaks at around 1700 nm, which are surely irrelevant to the samples and may result from the solvent, were cropped out in order to present clearly the absorption spectra of the samples.

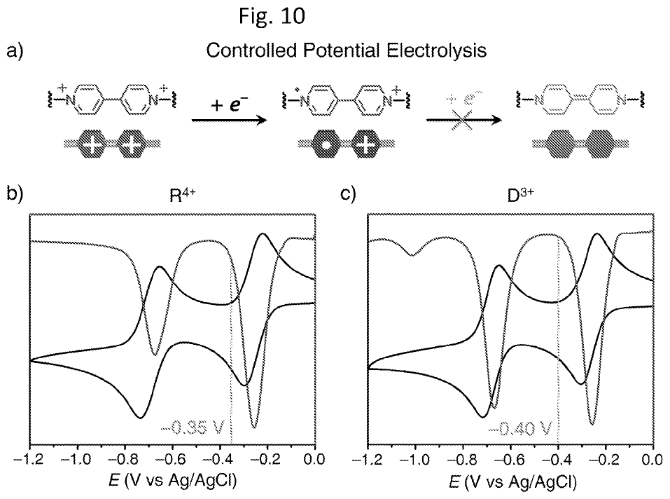

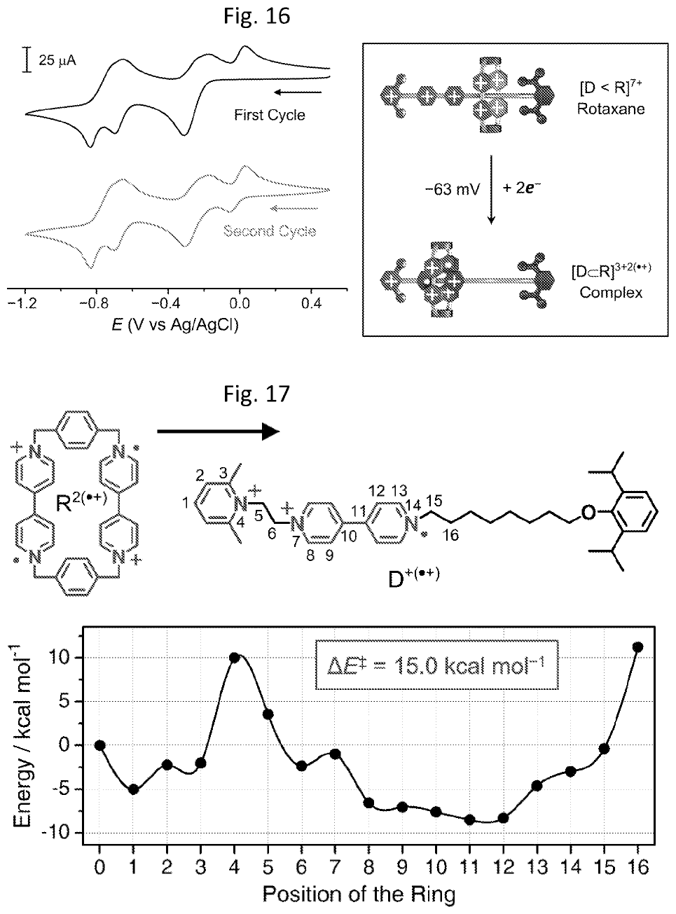

Cyclic voltammetry (CV) and differential pulse voltammetry (DPV) were carried out at room temperature in N₂-purged acetonitrile (MeCN) solutions with Gamry Multipurpose instrument (Reference 600) interfaced to a PC. A three-electrode system was used to record the data, in which the working electrode was a glassy carbon (0.071 cm²), the counter electrode was a Pt wire, and the reference electrode was a Ag/AgCl electrode. The surface of working electrode was polished routinely with 0.05 μm alumina-water slurry on a felt surface immediately before use. Tetrabutylammonium hexafluorophosphate (TBAPF₆) was used as supporting electrolyte with the concentration of 0.1 M. The concentrations of all the samples were set to 1.0 mM. In CV experiments, the scan rate was set to 100 mV s⁻¹ unless stated otherwise, and each measurement included three cycles of scan. The CV data in FIGS. 10, 14 and 15 are taken from the first cycle of scan, while FIG. 16 shows both the first and second cycles of scan.

Electron paramagnetic resonance (EPR) measurements at X-band (9.5 GHz) were performed with a Bruker Elexsys E580, equipped with a 4122SHQE resonator. Samples were contained in quartz tubes with I.D. 1.50 mm and O.D. 1.80 mm, sealed with a clear ridged UV curing epoxy (IllumaBond 60-7160RCL) and used immediately after preparation in a N₂-filled glovebox. Scans were performed with magnetic field modulation amplitude of 1 G and non-saturating microwave power of 1.5 mW. The results are the average of 64 scans.

### 2. Synthetic Protocols

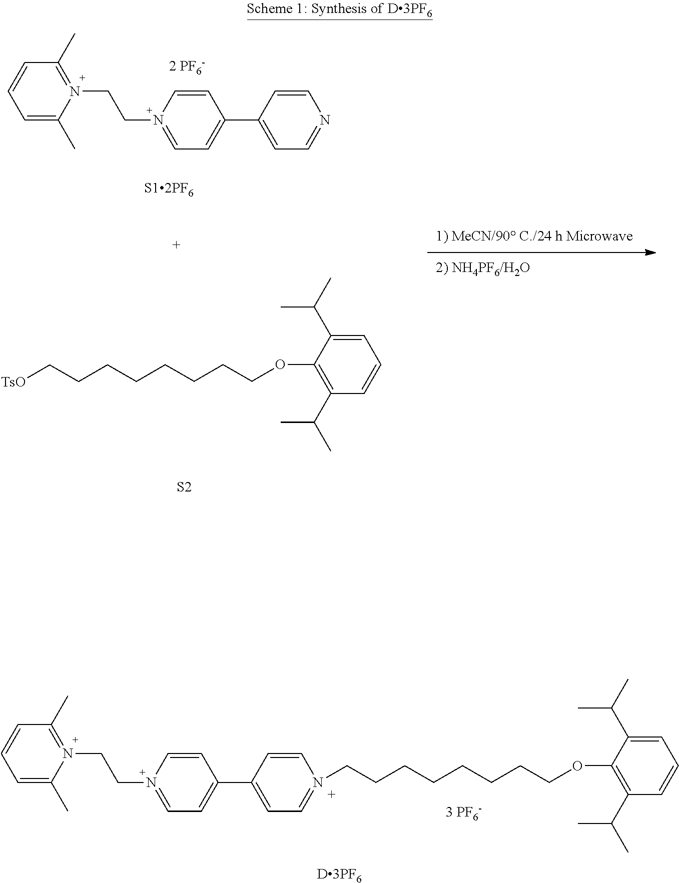

D•3PF₆: A mixture of S1•2PF₆ (630 mg, 1.1 mmol) and S2 (500 mg, 1.1 mmol) was stirred in 10 mL acetonitrile (MeCN) at 90° C. for 24 h and then cooled to room temperature. Precipitation was observed during the reaction. After removing the solvent under vacuum, the residue was purified using reversed-phase flash chromatography (C18: H₂O/MeCN with 0.1% TFA). Counterion exchange from TFA⁻ to PF₆⁻ by treating the product-containing fraction with excess of ammonium hexafluorophosphate (NH₄PF₆) generated a precipitate, which was collected by filtration, washed with H₂O for three times and dried under vacuum to afford D•3PF₆ as a white solid (655 mg, 60%). ¹H NMR (500 MHz, CD₃CN): δ 8.99 (d, J=6.7 Hz, 2H), 8.93 (d, J=6.6 Hz, 2H), 8.50 (d, J=6.7 Hz, 2H), 8.43 (d, J=6.4 Hz, 2H), 8.33 (t, J=7.9 Hz, 1H), 7.83 (d, J=7.9 Hz, 2H), 7.03-7.15 (m, 3H), 4.99-5.09 (m, 4H), 4.64 (t, J=7.6 Hz, 2H), 3.72 (t, J=6.4 Hz, 2H), 3.31 (hept, J=6.9 Hz, 2H), 2.86 (s, 6H), 2.01-2.10 (m, 2H), 1.76-1.85 (m, 2H), 1.49-1.59 (m, 2H), 1.39-1.47 (m, 6H), 1.19 (d, J=6.9 Hz, 12H). 13C NMR (126 MHz, CD₃CN): δ 157.6, 154.3, 152.4, 150.3, 147.4, 147.1, 146.6, 142.7, 129.7, 128.7, 128.3, 125.4, 124.9, 75.5, 63.1, 57.7, 51.7, 31.9, 30.9, 29.8, 29.5, 27.0, 26.6, 26.5, 24.2, 22.0. HR-ESI-MS (m/z): calcd. for [C₃₉H₅₄F₁₈N₃OP₃—PF₆]⁺ 870.3545, found 870.3533.

Cat•6PF₆: The [2]catenane model compound, namely Cat•6PF₆, was synthesized using a radical-templated^{5,6} approach. S3•2PF₆ (52 mg, 0.094 mmol) and R•4PF₆ (113 mg, 0.10 mmol) were dissolved in degassed MeCN (30 mL) in a N₂-filled glovebox. After adding excess of copper dust, the solution turned gradually from colourless to a dark purple colour, indicating the reduction of the starting materials and the formation of the trisradical tricationic complex. The reaction mixture was stirred at room temperature for 3 h before the addition of S4 (24 mg, 0.095 mmol) and N,N,N′,N″,N″-pentamethyldiethylenetriamine (PMDETA, 0.1 mL, excess). The reaction was conducted at room temperature for another 6 days, followed by the removal of copper dust by filtration and then the oxidation with excess of nitrosonium hexafluorophosphate (NOPF₆). After removing the solvent under vacuum, the crude product was purified using reversed-phase flash chromatography (C18: H₂O/MeCN with 0.1% TFA). Counterion exchange from TFA⁻ to PF₆—by treating the product-containing fraction with excess of NH₄PF₆ generated a precipitate, which was collected by centrifugation, washed with H₂O for three times and dried under vacuum to afford Cat•6PF₆ as a white solid (54 mg, 30%). ¹H NMR (500 MHz, CD₃CN): δ 9.09-9.00 (m, 12H), 8.36 (d, J=6.5 Hz, 8H), 8.07 (d, J=6.7 Hz, 4H), 7.92 (s, 2H), 7.17 (s, 8H), 5.66 (s, 8H), 5.07 (t, J=5.6 Hz, 4H), 4.44 (t, J=6.2 Hz, 4H), 3.65 (t, J=5.5 Hz, 4H), 1.66-1.54 (m, 4H), 0.74-0.61 (m, 4H), 0.22-0.08 (m, 4H), −1.39-−1.51 (m, 4H), −1.52-−1.62 (m, 4H). ¹³C NMR (126 MHz, CD₃CN): δ 148.9, 148.8, 147.6, 146.6, 143.9, 136.6, 131.1, 127.9, 126.8, 125.2, 65.3, 61.9, 50.6, 30.1, 29.7, 29.5, 28.9, 27.3, 26.3. HR-ESI-MS (m z): calcd. for [C₆₆H₇₄N₁₂P₆F₃₆—2PF₆]²⁻ 807.2358, found 807.2355.

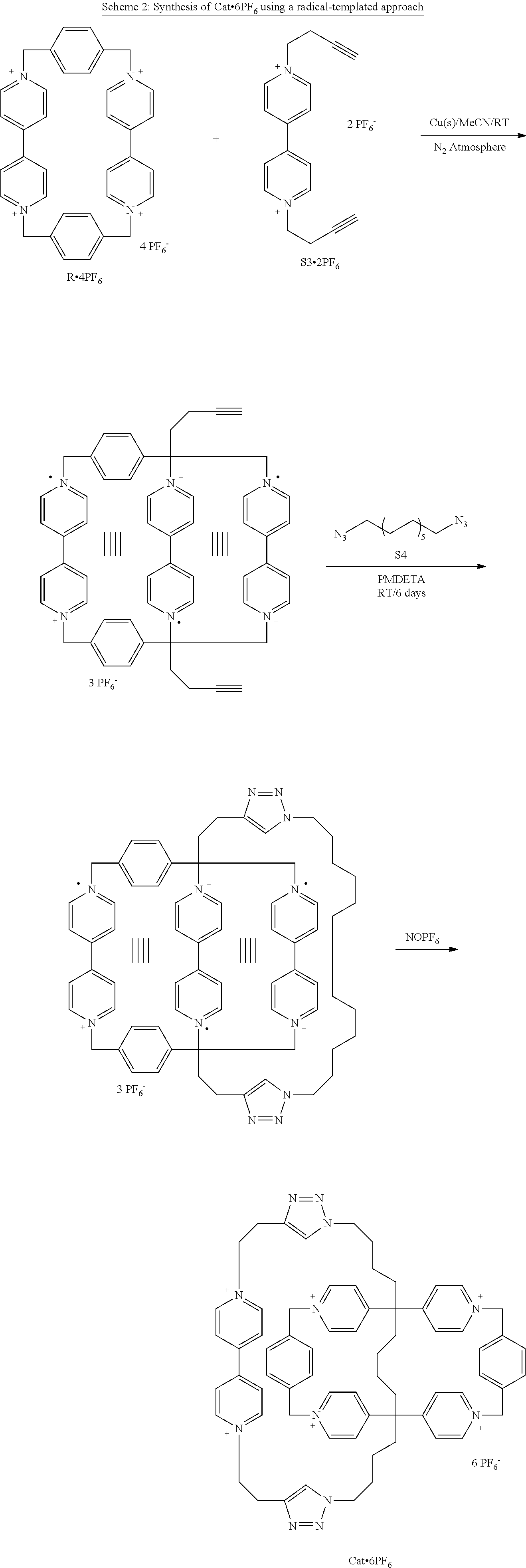

3. Preparation of R^{2(•+)} and D^{+(•+)}

The starting materials of the molecular recognition, R^{2(•+)} and D^{+(•+)}, were prepared by reducing the BIPY²⁺ units in their parent compounds (R⁴⁺ and D³⁺) to BIPY^{•+} radical cations. Given the fact that the kinetics of the molecular recognition process is very sensitive to additional electrons, the BIPY units in this host-guest system should be entirely in the radical cationic state (BIPY^{•-}), without the existence of either dicationic (BIPY²⁺) or neutral (BIPY⁽⁰⁾) states. It requires a complete single-electron reduction of BIPY²⁺ units while avoiding over-reduction.

TABLE 1
Comparison of different reduction methods used for
producing BIPY^{●+} radical cations

| | Complete | | |
|---|---|---|---|
| | Reduction | No Generation | |
| Reduction Methods | of BIPY²⁺ | of BIPY⁽⁰⁾ | Product(s) |
| Cu | x | ∘ | BIPY^{●+} + BIPY²⁺ |
| Zn | ∘ | x | BIPY^{●+} + BIPY⁽⁰⁾ |
| CoCp₂ (<1 eq) | x | ∘ | BIPY^{●+} + BIPY²⁺ |
| CoCp₂ (>1 eq) | ∘ | x | BIPY^{●+} + BIPY⁽⁰⁾ |
| Electrochemistry | ∘ | ∘ | BIPY^{●+} |

Different reduction methods of BIPY²⁺ units were screened, and their outcomes were compared and summarized in Table 1. When using copper (Cu) as the reductant, the single-electron reduction is not complete, generating a mixture of BIPY^{•+} and BIPY²⁺. In contrast, the use of zinc (Zn) results^{7,8} in the over-reduction, giving a mixture of BIPY^{•+} and BIPY⁽⁰⁾. Although a homogeneous, strong reductant, such as cobaltocene (CoCp₂), can induce a quantitative reduction of BIPY²⁺ units, it is difficult in practice to control the amount of CoCp₂ to be exactly 1 molar equivalent. Finally, we selected the controlled potential electrolysis⁹ (CPE) as an appropriate reduction method to produce BIPY^{•+} radical cations, because this approach is easy to operate, and its outcome is controllable by adjusting the working potential.

The CPE experiments were performed inside a N₂-filled glovebox using a custom-built H-cell electrolysis apparatus^{8,10}. A three-electrode system was used, which included a reticular vitreous carbon (RVC) working electrode, an RVC counter electrode and an Ag/AgCl reference electrode. The two RVC electrodes were placed into individual half-cell chambers (100 mL) that were separated by an ionic exchange membrane (Fumasep FAPQ-375-PP from Fuel Cell Store) and held together by a clamp. The Ag/AgCl reference electrode was inserted inside the working cell. The whole apparatus was connected to a Gamry multipurpose instrument (Reference 600) interfaced to a PC. The experimental parameters were instructed using the software of Gamry Framework Version 6.30 under the chronocoulometry mode.

The solid of R•4PF₆ (19.8 mg) or D•3PF₆ (18.3 mg) was dissolved in the MeCN solution of TBAPF₆ (0.05 M, 60 mL), and then transferred into the working cell. The counter cell was filled with a MeCN solution of (trimethylammonium methyl)ferrocene hexafluorophosphate, which served as the sacrificial electron donor. The working potentials for the electrolysis of R⁴ and D³⁺ were set as −0.35 and −0.40 V, respectively, according to their CV and DPV data (FIG. 10). The solutions were electrolysed under vigorous stirring (800 rpm) at room temperature for 40 min. The resulting stock solutions (0.3 mM) of R^{2(•+)} and D^{+(•+)} were stored in the N₂-filled glovebox for the use in subsequent experiments. It is noteworthy that the use of CPE method for electrolysis renders it practically difficult to achieve 100% conversion from BIPY²⁺ units to BIPY^{•+} radical cations. Specifically, there was ca. 1 mol % residual BIPY²⁺ in the solution after 40 min electrolysis. Therefore, 1 mol % CoCp₂ was added to both the electrochemically prepared solutions of R^{2(•+)} and D^{+(•+)} in prior to the kinetic measurements of the molecular recognition process.

### 4. Kinetic Studies on the Molecular Recognition

Molecular recognition was conducted by combining the electrochemically prepared R^{2(•+)} and D^{+(•+)}. The process was monitored by UV/Vis/NIR spectroscopy, because the [D⊂R]^{+3(•+)} triradical complex—the product of this molecular recognition—displays a characteristic¹¹⁻¹³ NIR absorption band at 1080 nm. In a typical procedure, a screw cap cuvette was charged with 500 μL of R^{2(•+)} and 500 μL of D^{+(•+)} (both are 0.3 mM in MeCN) under the N₂ atmosphere. Different additives were added to the solution if needed. After quick mixing, the solution was transferred to the spectrometer to record the UV/Vis/NIR spectra with time.

### 4.1 Long-Time Kinetic Measurement

Although the molecular recognition between R^{2(•+)} and D^{+(•+)} is undetectable (FIG. 2a) by UV/Vis/NIR spectroscopy within 70 min, we monitored the process in an extended time scale up to 10 h. This long-time measurement revealed a detectable but very slow increase (FIG. 11) in the absorption band (λₘₐₓ=1080 nm) characteristic of the [D⊂R]^{3+(•+)} trisradical complex. The low absorbance at 1080 nm indicated that the molecular recognition was still far from reaching equilibrium even after 10 h. Notably, during the process, the absorption (λmax=600 nm) for uncomplexed BIPY^{•+} radical cations showed a clear decrease, possibly caused by the penetration of O₂ into the solution It has been reported⁴⁻¹⁷ that the BIPY^{•+} radical cation can undergo a reversible disproportionation (Scheme 3) to form BIPY²⁺ and BIPY⁽⁰⁾, although the equilibrium constant (K_{disp}) is as low as 10⁻⁷. Considering the mechanism of electron catalysis, we speculated that the trace amount of BIPY⁽⁰⁾—generated by the weak disproportionation of the BIPY^{•+} units in either R^{2(•+)} or D^{+(•+)}—is responsible for the slow host-guest complexation between R^{2(•+)} and D^{+(•+)}, as observed in the long-time kinetic measurement (FIG. 11).

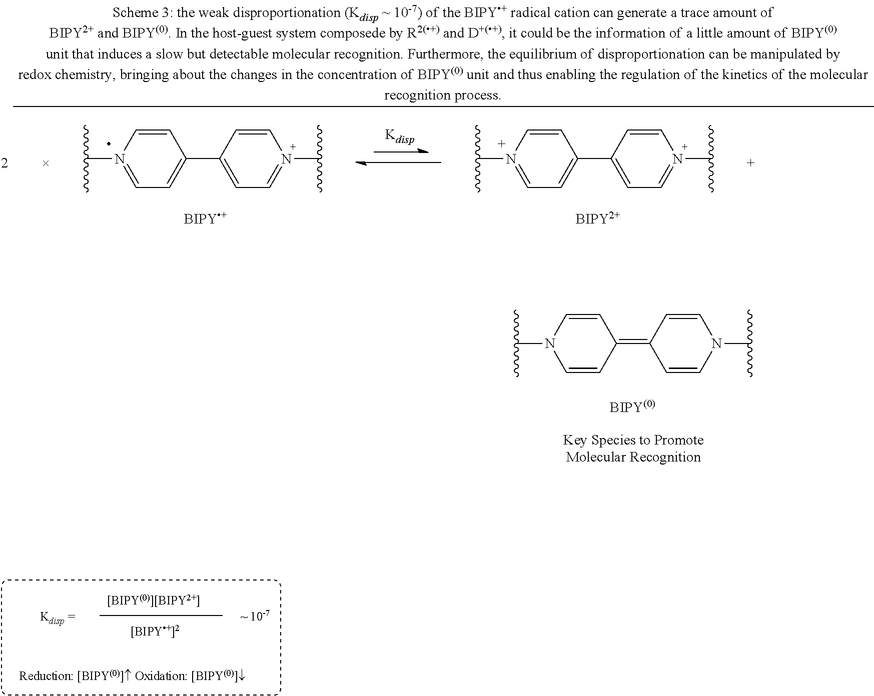

The equilibrium of disproportionation can be manipulated by redox chemistry. On the one hand, reduction—for example, by CoCp₂—will increase the concentration of BIPY⁽⁰⁾ unit, thereby accelerating (FIG. 2) significantly the molecular recognition process. On the other hand, oxidation can decrease the concentration of BIPY⁽⁰⁾ unit, which is expected to slow down the molecular recognition process.

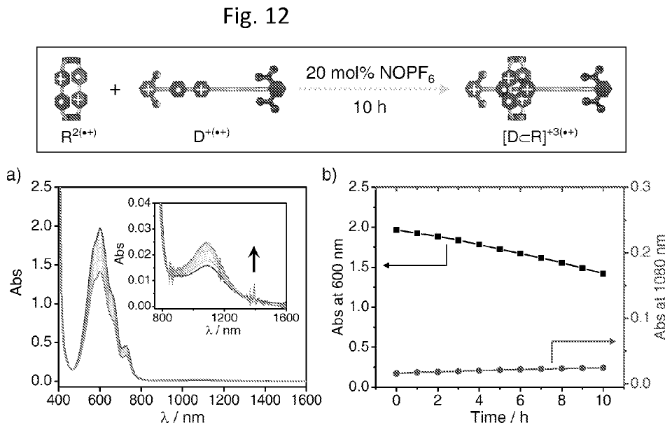

In an attempt to test this hypothesis, we introduced 20 mol % NOPF₆ (a chemical oxidant) into the equimolar mixture of R^{2(•+)} and D^{+(•+)}, and recorded the evolution of UV/Vis/NIR spectra. The molecular recognition between R^{2(•+)} and D^{+(•+)} under this condition was found to be slower (FIG. 12) than that without NOPF₆ (FIG. 11). Given the fact that the addition of 20 mol % NOPF₆ induces only a slight change in the concentrations of R^{2(•+)} and D^{+(•+)}, the distinct kinetics of the molecular recognition is probably a result of the decreased amount of BIPY⁽⁰⁾ unit. This result provides supporting evidence for the proposed effect of disproportionation on the molecular recognition process.

### 4.2 Estimation of the Binding Constant

Host-guest complexation between R^{2(•+)} and D^{+(•+)} is almost kinetically forbidden under ambient conditions, rendering it difficult to determine the binding constant by using traditional titration methods. Since the addition of a little amount (e.g., 4 mol %) of CoCp₂ can accelerate the molecular recognition process and has only a slight influence on the thermodynamic equilibrium, we can estimate the binding constant between R^{2(•+)} and D^{+(•+)} using the experimental data under catalytic conditions.

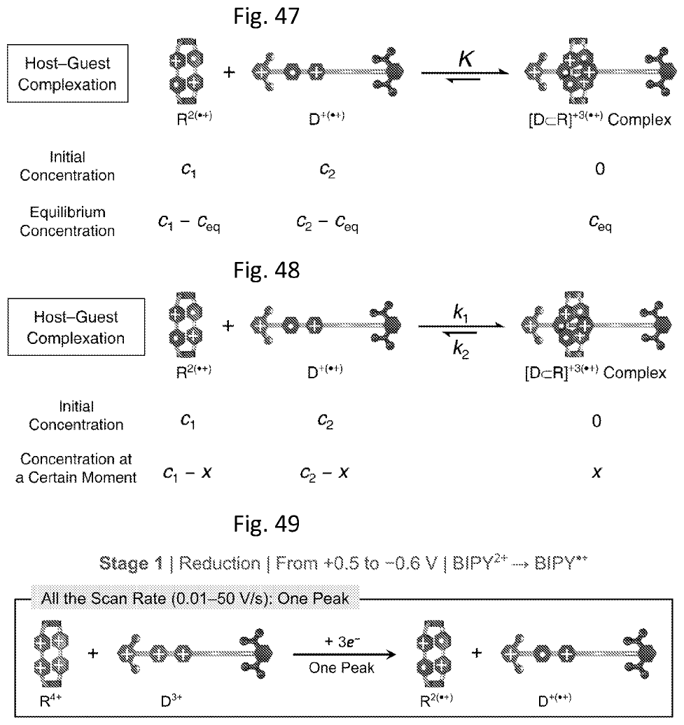

The reversible host-guest complexation can be written as the equation given in FIG. 47.

The binding constant K can be expressed as:

$$K = \frac{{c}_{eq}}{( {c}_{1} - {c}_{eq} ) ( {c}_{2} - {c}_{eq} )} \qquad ( 1 )$$

When introducing 4 mol % CoCp₂ into the host-guest system composed by 150 μM R^{2(•+)} and D^{+(•+)}, 6 μM of BIPY^{•+} radical cation was reduced to BIPY⁽⁰⁾ unit. As a result, the initial concentrations of R^{2(•+)} (c₁) and D^{+(•+)} (c₂) are 146 μM and 148 μM, respectively.

According to the Lambert-Beer's law:

$${A}_{1080 , eq} = {ε}_{1080} \cdot l \cdot {c}_{eq} \qquad ( 2 )$$

where A_{1080,eq} is the absorbance of the solution at 1080 nm after reaching equilibrium, and ε₁₀₈₀ is the molar absorption coefficient of the [D⊂R]^{+3(•+)} trisradical complex at 1080 nm, which can be estimated to be 1.12×10⁴ M⁻¹ cm⁻¹ from the literature¹³. l is the path length and set as 0.4 cm throughout this research. Therefore, the equilibrium concentration (c_{eq}) of the [D⊂R]^{+3(•+)} trisradical complex was calculated to be 56.1 μM.

From the values of c₁, c₂ and c_{eq}, the binding constant between R^{2(•+)} and D^{+(•+)} was calculated to be 6.8×10³ M⁻¹, which is comparable to the values reported¹³ in the literature.

### 4.3 Estimation of the Rate Constant

The rate constant of the molecular recognition between R^{2(•+)} and D^{+(•+)} was estimated from the kinetic data measured by UV/Vis/NIR spectroscopy. We employed the kinetic model of a two-to-one reversible reaction to deduce the rate equation (FIG. 48).

The forward rate (r) of this process can be expressed as:

$$r = \frac{dx}{dt} = {k}_{1} ( {c}_{1} - x ) ( {c}_{2} - x ) - {k}_{2} x \qquad ( 3 )$$

wherein c₁ and c₂ are the initial concentrations of R^{2(•+)} and D^{+(•+)}, respectively, x represents the concentration of the [D⊂R]^{+3(•+)} trisradical complex at a certain moment, and k₁ and k₂ refer to the rate constants of the association and dissociation, respectively. As a result of the relationship between k₁, k₂ and the equilibrium constant K:

$$K = \frac{{k}_{1}}{{k}_{2}} \qquad ( 4 )$$

the equation (3) can be transformed to be:

$${\frac{dx}{dt} = {[ x - \frac{1}{2} ( {c}_{1} + {c}_{2} + \frac{1}{K} ) ]}^{2} - \frac{1}{4} [ {c}_{1} - {c}_{2} )}^{2} + \frac{2 ( {c}_{1} + {c}_{2} )}{K} + \frac{1}{{K}^{2}} ] \qquad ( 5 )$$

In order to simplify the equation, we defined two constants m and n as follows:

$$m = \frac{1}{2} ( {c}_{1} + {c}_{2} + \frac{1}{K} ) \qquad ( 6 )$$

$$n = \frac{1}{2} \sqrt{{( {c}_{1} - {c}_{2} )}^{2} + \frac{2 ( {c}_{1} + {c}_{2} )}{K} + \frac{1}{{K}^{2}}} \qquad ( 7 )$$

By this way, the equation (5) was transformed to be:

$$\frac{dx}{dt} = {k}_{1} [ {( x - m )}^{2} - {n}^{2} ] \qquad ( 8 )$$

and then solved:

$$\frac{1}{2 n} [ ln ( m + n - x ) - ln ( m - n - x ) ] = {k}_{1} t + Q \qquad ( 9 )$$

wherein Q, a constant related to the data at t=0, is out of our concern.

Since the characteristic absorption for the [D⊂R]^{+3(•+)} trisradical complex at 1080 nm follows the Lambert-Beer's law:

$${A}_{1080} = {ε}_{1080} \cdot l \cdot x \qquad ( 10 )$$

we finally obtained the relationship between the absorbance at 1080 nm (A₁₀₈₀) and time (t) as follows:

$$\frac{1}{2 n} [ ln ( m + n - \frac{{A}_{1080}}{{ε}_{1080} \cdot l} ) - ln ( m - n - \frac{{A}_{1080}}{{ε}_{1080} \cdot l} ) ] = y = {k}_{1} t + Q \qquad ( 11 )$$

wherein a parametery was defined to simplify the form, which can be calculated from the data of A₁₀₈₀ together with a series of constants including ε₁₀₈₀, l, c₁, c₂ and K.

Fitting the data of A₁₀₈₀ to the time (t), we determined (FIG. 13) the rate constants (k₁) of the molecular recognition between R^{2(•+)} and D^{+(•+)} under different conditions. The values of k₁ have been summarized in Table 2.

TABLE 2
The values of c₁, c₂ and k₁ for the molecular
recognition processes between R^{2(●+)} and
D^{+(●+)} under different conditions

| Conditions | c₁/μM | c₂/μM | k₁/M⁻¹ s⁻¹ |
|---|---|---|---|
| Without additive | 150 | 150 | 0.00744 |
| 20 mol % NOPF₆ | 130 | 140 | 0.00296 |
| 1 mol % CoCp₂ | 149 | 149.5 | 0.200 |
| 2 mol % CoCp₂ | 148 | 149 | 0.526 |
| 4 mol % CoCp₂ | 146 | 148 | 4.74 |
| 8 mol % CoCp₂ | 142 | 146 | 24.2 |

### 4.4 Estimation of the Turnover Number

Based on the kinetic data obtained by UV/Vis/NTR spectroscopy, we calculated the turnover number (TON) of the electron-catalysed molecular recognition between R^{2(•+)} and D^{+(•+)}. During the kinetic measurements, O₂ was found to penetrate slowly into the solution and oxidize the BIPY⁽⁰⁾ units. As a result, the catalytic effect of electrons is gradually weakened and even quenched as time goes on. This happening, which is non-negligible for long-time molecular recognition processes catalysed by a small number of electrons, renders it difficult to determine accurately the TON value. Therefore, a lower limit of TON was estimated using the yield of the [D⊂R]^{+3(•+)} trisradical complex after a moderate time (70 min), according to the following formula:

$$TON = \frac{c ( Com )}{c ( {e}^{-} )} = \frac{{A}_{1080 , 70 min}}{{ε}_{1080} \cdot l \cdot c ( {e}^{-} )} \qquad ( 12 )$$

wherein c(Com) is the concentration of the [D⊂R]^{+3(•+)} trisradical complex at 70 min and can be determined from the absorbance of the solution at 1080 nm, while c(e⁻), the molar concentration of injected electrons, is approximately equal to that of CoCp₂. The estimated TON values under different conditions are summarized in Table 3.

TABLE 3
The estimated values of the turnover number (TON)
for the electron-catalysed molecular recognition
processes under different conditions

| Conditions | c(Com)/μM | c(e⁻)/μM | TON |
|---|---|---|---|
| 1 mol % CoCp₂ | 19.5 | 1.5 | 13 |
| 2 mol % CoCp₂ | 33.5 | 3.0 | 11 |
| 4 mol % CoCp₂ | 56.1 | 6.0 | 9 |
| 8 mol % CoCp₂ | 54.9 | 12 | 5 |

In the case of 4 and 8 mol % CoCp₂, the TON values are relatively small, because the yield of the [D⊂R]^{+3(•+)} trisradical complex is limited by the thermodynamic equilibrium between substrates and complexes. In the case of 1 mol % CoCp₂, the TON was estimated to be 13, i.e., each mole of electrons is able to catalyse the formation of at least 13 moles of the [D⊂R]^{+3(•+)} trisradical complex. The modest value of TON, not only reflects the quenching effect by O₂, but also results from the excessive stability of the catalytic intermediate, i.e., the [D⊂R]^{+2(•+)} bisradical complex, which inhibits the completion of catalytic cycles. Further efforts need to be made in order to develop more efficient electron-catalysed molecular recognition systems involving less stable intermediates, so as to increase the TON value.

### 5. Electron Catalysis Evidenced by Cyclic Voltammetry

In addition to UV/Vis/NIR spectroscopy, the electron-catalysed molecular recognition between R^{2(•+)} and D^{+(•+)} has also been evidenced by cyclic voltammetry (CV). The CV scan of either R⁴⁺ or D³⁺ indicated two sequential, reversible reductions from BIPY²⁺ units to BIPY^{•-} radical cations and further to BIPY⁽⁰⁾ units. Specifically, the reduction peaks of R⁴⁺ (FIG. 14a) at −297 and −736 mV can be ascribed to the reduction from R⁴⁺ to R^{2(•+)} and from R^{2(•+)} to R), respectively. Likewise, the two-step reduction of D³⁺ (FIG. 14b) occurred at −306 and −717 mV successively.

In contrast with the relatively simple redox behaviours of R⁴⁺ or D³⁺ alone, the CV scan of the mixture of R⁴⁺ and D³⁺ from +0.5 to −1.2 V displayed (FIG. 14c) a complicated and irreversible trace. In particular, the negatively shifted reduction peak at −835 mV and the positively shifted oxidation peak at +30 mV, compared with R⁴⁺ or D³⁺ alone, are characteristic¹² of the formation of the [D⊂R]^{+3(•+)} trisradical complex because the radical-pairing interactions involved in the complex can stabilize the BIPY^{•+} radical cations against (1) being reduced to BIPY⁽⁰⁾ and (2) being oxidized to BIPY²⁺.

The oxidation peak at +30 mV characteristic for the [D⊂R]^{+3(•+)} trisradical complex, however, was found to disappear, when performing a short-range CV scan (FIG. 14e) of the mixture of R⁴⁺ and D³⁺ from +0.5 to −0.6 V. Under this set of conditions, the BIPY²⁺ units were only reduced to BIPY^{•+} radical cations before reoxidation, meaning that no generation of BIPY⁽⁰⁾ units occurs during the process. The clear difference, therefore, between the CV traces with different scanning ranges indicates that the reduction from BIPY^{•+} radical cations to BIPY⁽⁰⁾ units—i.e., the injection of additional electrons—is essential for promoting the molecular recognition between R^{2(•+)} and D^{+(•+)}. When performing a middle-range CV scan (FIG. 14d) on a mixture of R⁴⁺ and D³⁺ from +0.5 to −0.75 V, we still observe the relevant peak at +30 mV, albeit with decreased intensity. This control experiment has excluded the possibility that the formation of the [D⊂R]^{+3(•+)} trisradical complex originates from the reduction process at −835 mV.

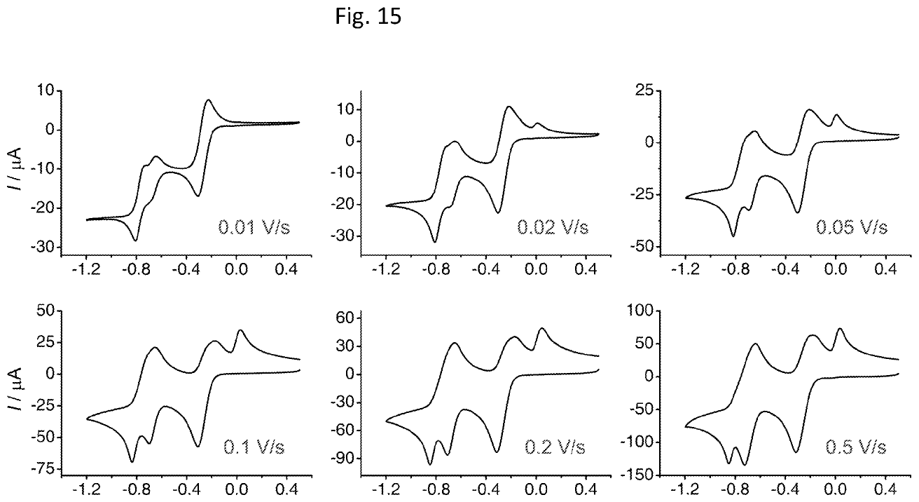

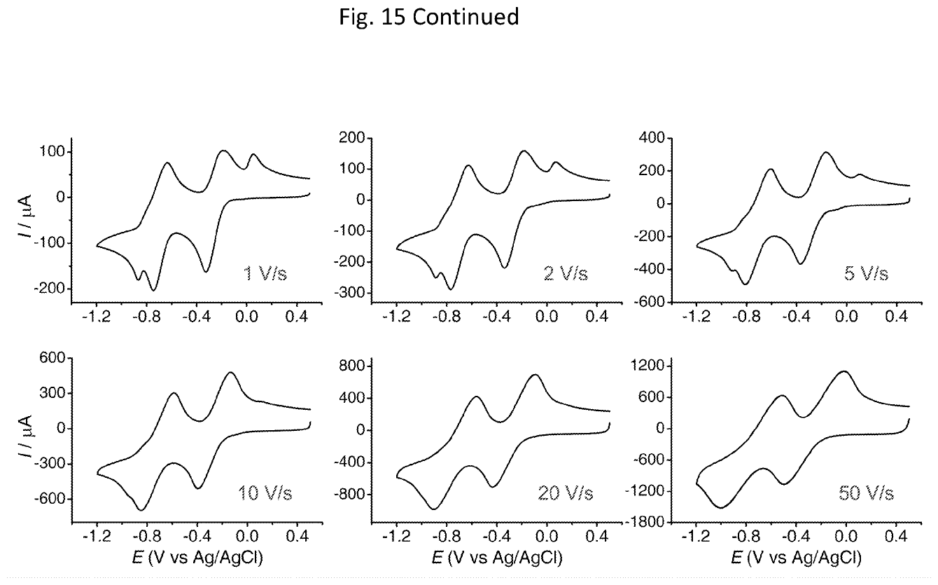

In order to reveal the influence of electron-catalysed molecular recognition on the redox properties of the host-guest system, we performed (FIG. 15) CV measurements on the mixture of R⁴⁺ and D³⁺ at variable scan rates. The CV traces were found to be highly dependent on the scan rates, suggesting that a correlation exists between supramolecular association/dissociation and electron transfer events. By analysing the CV data at different scan rates and referring to the literature¹⁻¹³, a whole redox cycle was divided into four stages and all the events occurring during the process were illustrated in Schemes 4-7 (FIGS. 49-52).

Stage 1 (Scheme 4, FIG. 49), scanning from +0.5 to −0.6 V, results in the reduction of BIPY²⁺ units to BIPY^{•+} radical cations. In this stage, R⁴⁺ and D³⁺ are reduced to R^{2(•+)} and D^{+(•+)} respectively, at the same potential. Thus, only one reduction peak is observed regardless of the scan rate. Note that the complexation between R^{2(•+)} and D^{+(•+)} is kinetically forbidden until more electrons are injected in Stage 2.

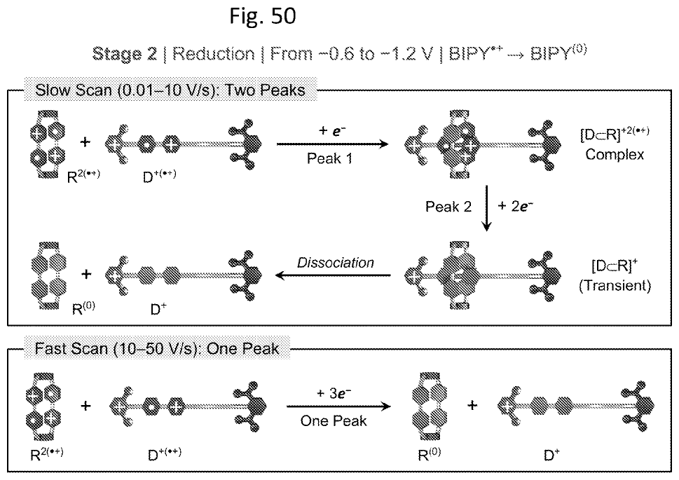

Stage 2 (Scheme 5, FIG. 50), scanning from −0.6 to −1.2 V, results in the reduction of BIPY^{•+} radical cations to BIPY⁽⁰⁾ neutral units. When the scan rate is relatively slow (0.01-10 V/s), the mixture of R^{2(•+)} and D^{+(•+)} accepts three electrons gradually. Frist of all, the injection of two electrons leads to the formation of the [D⊂R]^{2(•+)} bisradical complex. Further reduction of all the BIPY units to their neutral states occurs at a more negative potential, indicating the stabilizing effect of the radical-pairing interaction on the bisradical complex. Finally, the lack of interactions between R^{(O)} and D⁺ induces their dissociation. Therefore, two reduction peaks have been observed in this stage for the slow CV scans. When the scan rate is very fast (10-50 V/s), however, the mixture of R^{2(•+)} and D^{+(•+)} accepts three electrons rapidly and generates directly R⁽⁰⁾ and D⁺, so that the formation of the bisradical complex cannot be detected. Therefore, only one reduction peak has been observed in this stage involving the fast CV scans.

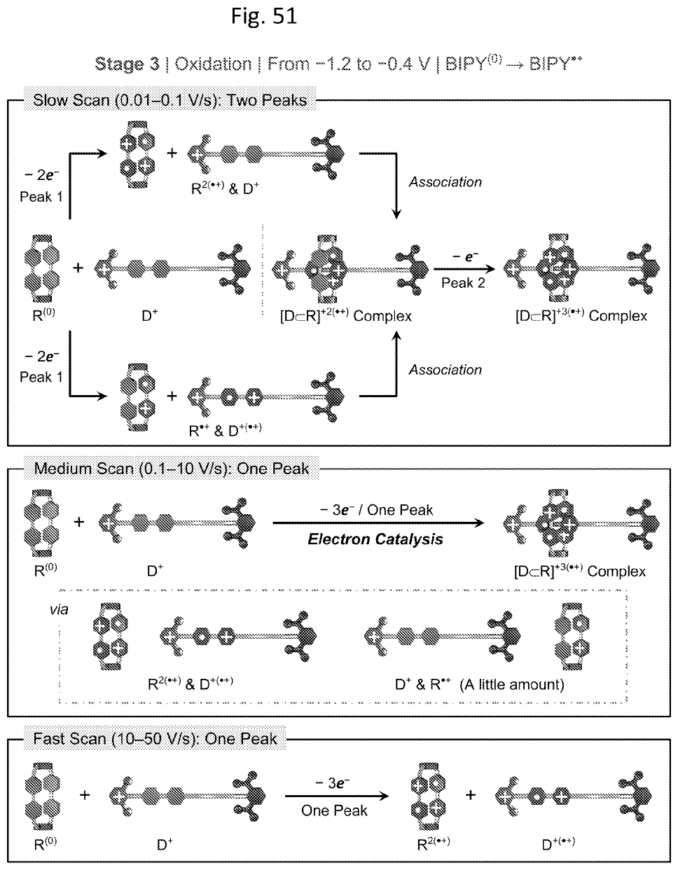

Stage 3 (Scheme 6, FIG. 51), scanning from −1.2 to −0.4 V, results in the reoxidation of BIPY⁽⁰⁾ neutral units to BTPY^{•+} radical cations. Three possible pathways are proposed for this stage:

When the scan rate is slow (0.01-0.1 V/s), the mixture of R⁽⁰⁾ and D⁺ has the chance to lose two electrons first of all, generating the couple of R^{2(•+)} and D⁺ or the couple of R^{•+} and D^{+(•+)}. Both couples can be associated into the [D⊂R]^{+2(•+)} bisradical complex. On account of the formation of this stable bisradical complex, the potential for the two-electron oxidation of the mixture of R⁽⁰⁾ and D⁺ is less positive than that required for the oxidation of R⁽⁰⁾ or D⁺ alone to its radical cation state. This two-electron oxidation peak is only observable using very slow scan rates because sufficient time is required to overcome the energy barrier for the assembly of the bisradical complex from its components. Subsequently, another peak at a more positive potential is observed and ascribed to oxidation of the [D⊂R]^{+2(•+)} bisradical complex to the [D⊂R]^{+2(•+)} trisradical complex. Thus, two reoxidation peaks have been observed in this stage for the slow CV scans, leading to the generation of the trisradical complex.

When the scan rate is in the medium (0.1-10 V/s) range, we cannot observe a considerate amount of the [D⊂R]^{+2(•+)} bisradical complex formed during the measurements. The mixture of R⁽⁰⁾ and D⁺ loses three electrons at a moderate speed, generating R^{2(•+)} and D^{+(•+)} as well as a small amount of R^{•+} and D⁺. The latter two species will initiate the electron-catalysed molecular recognition between R^{2(•+)} and D^{+(•+)}, affording the [D⊂R]^{+3(•+)} trisradical complex. Thus, although only one reoxidation peak has been observed for the medium-rate CV scans, the trisradical complex is still generated in this stage.

When the scan rate is fast (10-50 V/s), the mixture of R⁽⁰⁾ and D⁺ loses three electrons in an instantaneous manner. Hence, only one reoxidation peak has been observed in this stage for the fast CV scans, with neither the [D⊂R]^{+2(•+)} bisradical complex nor the [D⊂R]^{+3(•+)} trisradical complex being generated.

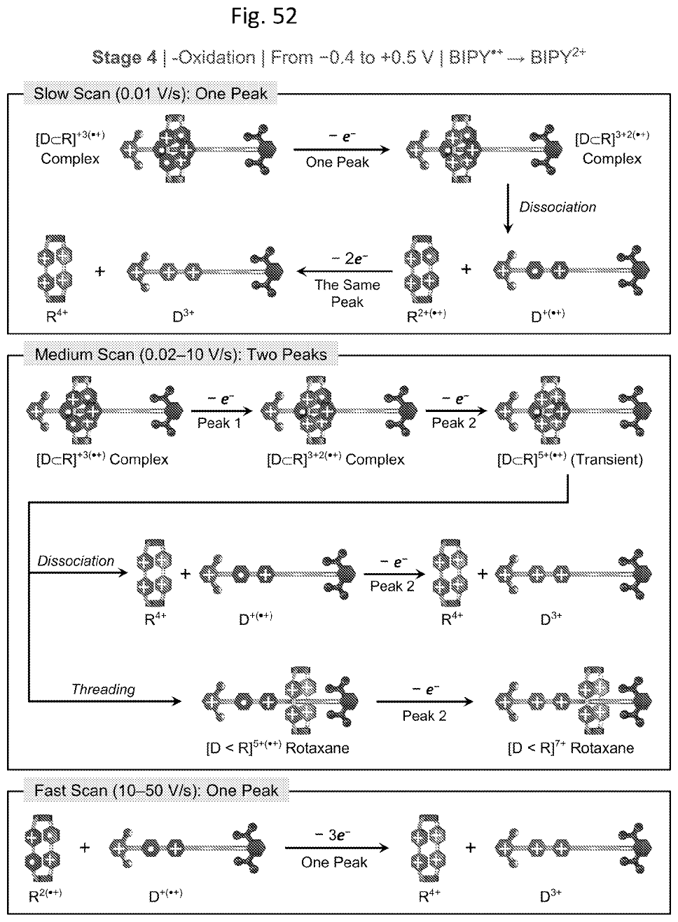

Stage 4 (Scheme 7, FIG. 52), scanning from −0.4 to +0.5 V, results in the reoxidation from BIPY^{•+} radical cations to BIPY²⁺ units. Three possible pathways are proposed for this stage:

When the scan rate is slow (0.01 V/s), the [D⊂R]^{+3(•+)} trisradical complex generated in Stage 3 loses one electron first of all, forming a [D⊂R]^{3+(•+)} bisradical complex composed of two BIPY^{•+} radical cations and one BIPY²⁺ unit. When the CV scan is performed very slowly, this weakly bounded complex—as a result of increased Coulombic repulsion—has sufficient time to undergo dissociation and generate R^{2+(•+)} and D^{+(•+)}. At the same potential, the resulting species lose two electrons to afford R⁴⁺ and D³⁺. Therefore, only one reoxidation peak is observed in this stage for the slow CV scan.

When the scan rate is in the medium (0.01-10 V/s) range, the [D⊂R]^{+3(•+)} trisradical complex generated in Stage 3 loses one electron first of all, forming a [D⊂R]^{3+2(•+)} bisradical complex composed of two BIPY^{•+} radical cations and one BIPY²⁺ unit. The oxidation of this complex is less favourable than that of the trisradical complex on account of the increased number of positive charges. Hence, when the CV scan is faster than the dissociation of the [D⊂R]^{3+2(•+)} bisradical complex, the complex will lose another electron at a more positive potential and produce a [D⊂R]^{5+(•+)} monoradical complex. Upon the formation of this transient complex, Coulombic repulsion is further increased, leading to (1) the dissociation of the complex to R⁴⁺ and D^{+(•+)}, or (2) the threading of the ring onto the collecting oligomethylene chain in the guest molecule, thus forming a [D<R]^{5+(•+)} rotaxane. Here the symbol “<” denotes that the ring encircles the dumbbell in a rotaxane. Either the mixture of R⁴⁺ and D^{+(•+)} or the [D<R]^{5+(•+)} rotaxane can be oxidized, at the same potential, to the mixture of R⁴⁺ and D³⁺ or the [D<R]⁷⁺ rotaxane, respectively. Hence, the medium-rate CV scans in this stage give rise to two possible sets of products, both of which result from two reoxidation peaks.

When the scan rate is fast (10-50 V/s), the products in Stage 3 are R^{2(•+)} and D^{+(•+)}, both of which lose electrons independently at the same potential and restore R⁴⁺ and D³⁺. Thus, only one reoxidation peak has been observed in this stage for the fast CV scans.

After one cycle of a middle-rate (e.g., 100 mV s⁻¹) CV scan on the mixture of R⁴⁺ and D³⁺, some of the starting materials have been transformed to the [D<R]⁷⁺ rotaxane. As a result, the second cycle of the CV scan displays (FIG. 16) a new reduction peak at −63 mV, compared with the first cycle of scan. This positively shifted peak can be ascribed to a two-electron reduction (FIG. 16) of the [D<R]⁷⁺ rotaxane to the [D⊂R]^{3+2(•+)} bisradical complex, considering that the intramolecular binding promotes the generation of two BIPY^{•+} radical cations in the rotaxane. 6. Mechanism of the Electron-Catalysed Molecular Recognition

### 6.1 Discussion on the Catalytic Pathways

The electron-catalysed molecular recognition between R^{2(•+)} and D^{+(•+)} is accomplished in two stages: (1) the formation of a key intermediate, the [D⊂R]^{+2(•+)} bisradical complex, by injecting electrons into the starting materials, and (2) the transformation of this intermediate into the final product, i.e., the [D⊂R]^{+3(•+)} trisradical complex. Since both R^{2(•+)} and D^{+(•+)} contain the BIPY^{•+} radical cation and can accept the injected electron, there should be more than one possible pathway during electron catalysis.

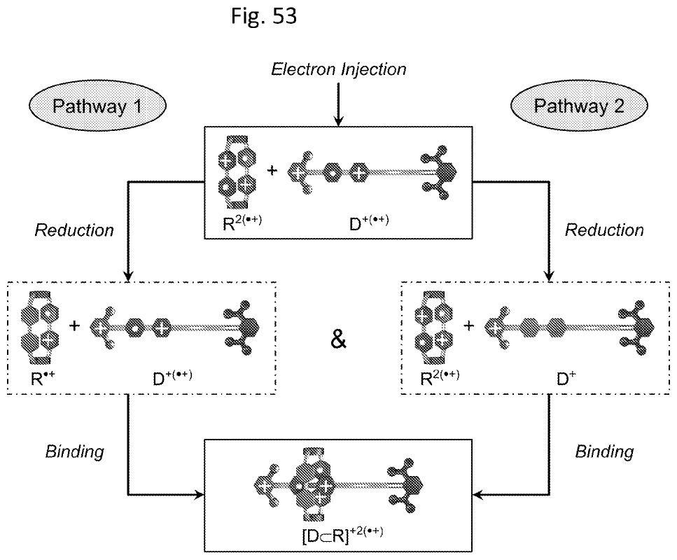

At the beginning, when an electron is injected into the system, two possible pathways (Scheme 8, FIG. 53) exist, depending on whether R^{2(•+)} or D^{+(•+)} accepts this electron.

In Pathway 1, one of the BIPY^{•+} radical cations in R^{2(•+)} is reduced to BIPY^{•+} unit, and the resulting R^{•+} can bind rapidly with D^{+(•+)} to afford the [D⊂R]^{+2(•+)} bisradical complex.

In Pathway 2, the BIPY^{•+} radical cation in D^{+(•+)} is reduced to BIPY⁽⁰⁾ unit, and the resulting D⁺ can bind rapidly with R^{2(•+)} to afford the [D⊂R]^{+2(•+)} bisradical complex.

Given the fact that the reduction potential (FIGS. 10 and 14) of D^{+(•+)} is slightly more positive than that of R^{2(•+)}, the formation of the R^{2(•+)}/D⁺ couple (i.e., Pathway 2) is relatively preferred. Both pathways decrease the number of positive charges in the system, and thereby lower the energy barrier associated with the binding between the ring and the dumbbell. See the DFT calculation results in Section 6.2. Since the electron is exchangeable, the R^{•+}/D^{+(•+)} couple (coming from Pathway 1) and the R^{2(•+)}/D⁺ couple (coming from Pathway 2) can co-exist in the system and undergo mutual transformation. Therefore, these two pathways are interconnected, leading jointly to the formation of the [D⊂R]^{+2(•+)} bisradical complex, the key intermediate in the electron-catalysed molecular recognition process.

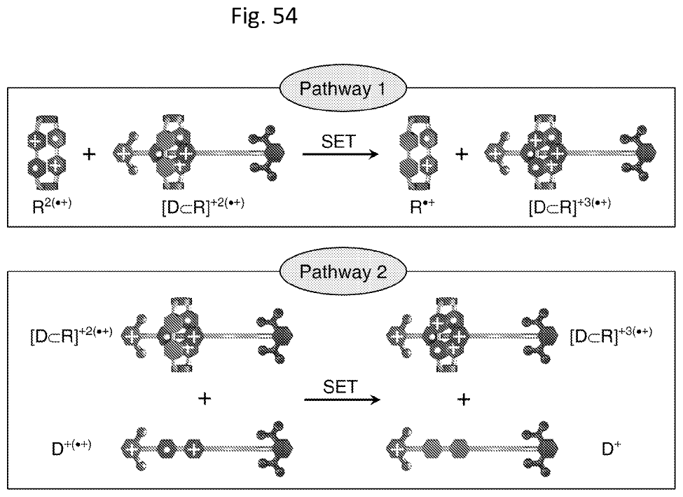

When it comes to the transformation from the [D⊂R]^{+2(•+)} bisradical complex to the [D⊂R]^{+3(•+)} trisradical complex, there are also two possible pathways (Scheme 9, FIG. 54).

In Pathway 1, single-electron transfer (SET) occurs between the [D⊂R]^{+2(•+)} bisradical complex and R^{2(•+)}, affording the [D⊂R]^{+3(•+)} trisradical complex and R**.

In Pathway 2, it is D^{+(•+)} that participates in the SET with the [D⊂R]^{+3(•+)} trisradical complex, generating the [D⊂R]^{+3(•+)} trisradical complex and D⁺.

Both pathways lead to the formation of the [D⊂R]^{+3(•+)} trisradical complex—the final product of the molecular recognition—and meanwhile, the regeneration of either R^{•+} or D⁺ to sustain the catalytic cycle.

In summary, all the possible pathways during the molecular recognition process can be unified as electron catalysis, with the electron serving as the actual catalyst.

### 6.2 Quantum Mechanical Calculations of the Energy Barriers

In order to elucidate the mechanism of the electron catalysis, we performed Quantum Mechanics (QM) calculations at the level of Density Functional Theory (DFT) to determine the energy barriers of the molecular recognition processes with or without injection of an electron. In these calculations, we optimized the geometries of molecules using Density Functional Theory at the M06-2X/6-31G* level¹⁸ using Jaguar¹⁹ v10.6 with the PBF Poisson-Boltzmann solvation model²⁰ based on acetonitrile (ε=37.5 and R₀=2.18 Å). These single-point optimized structures were refined using the larger basis for M06-2X/6-311++G** to obtain the final energy. We included two Cl⁻ anions to mimic the electrostatic effect of PF₆⁻ anions in the experimental system.

The introduction of explicit counterions into the PBF implicit solvation model decreases the net charge of the whole system, reducing uncertainty in the calculated solvation energy based on the implicit solvation model. For the multi-radical systems in this study, we found the open-shell unrestricted wavefunction with low spin to be the ground state or almost degenerate with the high spin state. The energy of the true ground state of low spin wavefunction was estimated²¹ by correcting the spin contamination to obtain the energy difference between the high-spin triplet and the low-spin singlet.

Calculations were carried out on three host-guest systems:
1) R^{2(•+)} and D^{+(•+)},
2) R^{•+} and D^{+(•+)}, and
3) R^{2(•+)} and D⁺.

For each system, the potential energy surface was obtained by scanning the centre of the ring (defined as the average position of four methylene carbon atoms) through the atoms (numbered as position 1 to 16) in the dumbbell, as shown in FIGS. 17-19. The position 0 corresponds to the infinite separation of two molecules (each surrounded by one Cl⁻ anion). The energy of this state is defined as zero.

In the potential energy surface obtained by DFT calculations, Coulombic repulsion between the ring and the dumbbell molecules contributes substantially to the energy barrier (ΔE^{‡}) during the threading process. As a result, the barrier heights differ substantially under different charge-number conditions.

In the R^{2(•+)}-D^{+(•+)} system (FIG. 17), we determined ΔE^{‡}=15.0 kcal mol⁻¹ from the energy difference between position 1 and 4. This large energy barrier arises from the Coulombic repulsion between R^{2(•+)} and D^{+(•+)}. Specifically, both the positively charged BIPY^{•+} unit and PY⁺ group in D^{+(•+)}—working in concert on account of their close proximity—contribute to the Coulombic repulsion with the two positively charged BIPY^{•+} units in R^{2(•+)}. Therefore, the energy barrier can decrease substantially by reducing the charge number of either R^{2(•+)} or D^{+(•+)}.

In the R^{•+}-D^{+(•+)} system (FIG. 18), we determined ΔE^{‡}=9.3 kcal mol⁻¹ from the energy difference between position 3 and 7, which is 5.7 kcal mol⁻¹ lower than that in the R^{2(•+)}-D^{+(•+)} system.

In the R^{2(•+)}-D⁺ system (FIG. 19), we determined ΔE^{‡}=8.8 kcal mol⁻¹ from the energy difference between position 3 and 5, which is 6.2 kcal mol⁻¹ lower than that in the R^{2(•+)}-D^{+(•+)} system.

These results indicate that the injection of an electron into either R^{2(•+)} or D^{+(•+)} can induce a remarkable decrease in the Coulombic repulsion, thereby decreasing the energy barrier for the molecular recognition process.

Practically, upon the injection of an electron, there forms a mixture of the R^{•+}-D^{+(•+)} couple and the R^{2(•+)}-D⁺ couple, which are interconverted through single electron transfer between the ring and the dumbbell molecules. The energy barrier for the electron transfer is governed by the reorganization energy involving the conformational change of the dumbbell molecule, the movement of counterions, and the polarization field imposed by solvent molecules. If this energy barrier is negligible compared with the energy barrier for the complexation between R^{•+} and D^{+(•+)} or between R^{2(•+)} and D⁺ (which means that the extra electron can “freely” jump to the more favourable site with little barrier), we can combine the potential energy surfaces of R^{•+}-D^{+(•+)} and R^{2(•+}-D⁺ systems to construct an overall potential energy surface for the molecular recognition associated with the injection of an electron.

We found that for molecular recognition in the presence of an extra electron, the R^{•+}-D^{+(•+)} couple is the energetically more favourable state or almost degenerate with the R^{2(•+)}-D⁺ couple. By comparing the electronic energy of these two couples and taking the lower value as the electronic ground state at each position, we obtained the overall potential energy surface for the binding process with the injection of an electron, as plotted in FIG. 20 together with the potential energy surface for the binding process without the injection of electron (i.e., the R^{2(•+)}-D^{+(•+)} system). The comparison between these two energy profiles further confirms that the introduction of an electron can decrease the energy barrier of the molecular recognition.

In order to estimate the change in Gibbs free energy (ΔG) of the molecular recognition with or without injection of an electron, the values of enthalpy change (ΔH) and entropy change (ΔS) need to be determined. Assuming that the volume change during this noncovalent binding event is small in solution, the enthalpy change approximately equals to the binding energy, i.e., ΔH≈ΔE. In order to estimate the entropy change, we computed the Hessian matrix (the second derivative of energy) of simplified model systems in which the ring molecule remained unchanged while the long alkyl chain and the 2,6-diisopropylphenyl terminal group in the dumbbell molecule were replaced with a propyl unit. The calculation of these model systems was performed in gas phase with no counterion. In contrast to species in the gas phase, species in the solution phase cannot translate or rotate freely, leading to liberational modes. To account for the solvent confinement, we scale the corresponding entropic contributions due to translation and rotation down by a factor²² of 0.5. The calculated values of ΔH, ΔS and ΔG for the three host-guest systems are summarized in Table 4.

TABLE 4
The calculated thermodynamic parameters ΔH, TΔS, and ΔG at 298
K for the molecular recognition in the three host-guest systems

| | R^{2(●+)} − D^{+(●+)} | R^{●+} − D^{+(●+)} | R^{2(●+)} − D⁺ |
|---|---|---|---|
| | System | System | System |
| ΔH/kcal mol⁻¹ | −8.5 | −13.2 | −6.6 |
| TΔS/kcal mol⁻¹ | −2.6 | −3.0 | −2.6 |
| ΔG_{298 K}/kcal mol⁻¹ | −5.9 | −10.2 | −4.0 |

TABLE 5
The charge population on the ring molecule at different positions
in the three host-guest systems. These are electrostatic potential
(ESP) derived charges using the M06-2X/6-311++G** level of DFT.

| | The charge population on the R^{2(●+)} or R^{●+} | | |
|---|---|---|---|
| | R^{2(●+)} − D^{+(●+)} | R^{●+} − D^{+(●+)} | R^{2(●+)} − D⁺ |
| Position | System | System | System |
| 0 | 2.02 | 1.00 | 1.95 |
| 1 | 2.13 | 0.94 | 1.92 |
| 2 | 2.15 | 0.97 | 1.98 |
| 3 | 2.21 | 0.96 | 1.80 |
| 4 | 2.27 | 1.03 | 2.00 |
| 5 | 2.29 | 1.22 | 2.53 |
| 6 | 2.16 | 1.35 | 2.39 |
| 7 | 1.89 | 1.12 | 2.27 |
| 8 | 1.81 | 0.91 | 2.18 |
| 9 | 1.94 | 0.88 | 1.96 |
| 10 | 1.95 | 0.91 | 1.74 |
| 11 | 1.91 | 1.07 | 1.89 |
| 12 | 2.03 | 1.06 | 1.99 |
| 13 | 2.01 | 0.94 | 2.02 |
| 14 | 2.27 | 0.83 | 2.00 |
| 15 | 2.07 | 0.89 | 1.99 |
| 16 | 2.02 | 0.82 | 2.04 |

### 6.3 Spectroscopic Detection of the Key Intermediate

[D⊂R]^{+2(•+)} bisradical complex, the key intermediate of the electron-catalysed molecular recognition process, was detected (FIG. 21) by UV/Vis/NIR spectroscopy. Although the addition of 4 mol % CoCp₂ gives rise to the catalytic formation of [D⊂R]^{+3(•+)} trisradical complex—as indicated by the absorption band at 1080 nm-increasing the amount of CoCp₂ from 4 to 100 mol % brings about the decrease of the absorption band at 1080 nm, together with the appearance of a new, red-shifted absorption band at 1700 nm. We ascribe this absorption band to the [D⊂R]^{+2(•+)} bisradical complex.

The SET between the [D⊂R]^{+2(•+)} bisradical complex and R^{2(•+)} or D⁺—a process leading to the formation of the [D⊂R]^{+3(•+)} trisradical complex—was probed by UV/Vis/NIR spectroscopy. When introducing R^{2(•+)} into the solution of [D⊂R]^{+2(•+)} bisradical complex, we observed (FIG. 24a) the decay of the absorption (λₘₐₓ=1700 nm) for the bisradical complex, accompanied with the increase of the absorption (λₘₐₓ=1080 nm) for the trisradical complex. Notably, more than 1 eq of R^{2(•+)} was required (FIG. 24b) to render this evolution being complete. This result is reasonable because the reduction potential of the [D⊂R]^{+3(•+)}/[D⊂R]^{+2(•+)} redox couple is close to (instead of much lower than) that of the R^{3(•+)}/R^{2(•+)} couple. Considering the fact that the electron is injected in a catalytic amount (e.g., 4 mol %) during the molecular recognition process, there is always excess of R^{2(•+)} in relation to the low concentration of [D⊂R]^{+2(•+)} bisradical complex, guaranteeing the sufficiency of the SET process and the sustainability of the catalytic cycle.

In a similar manner, we have also demonstrated (FIG. 25) experimentally the SET between the [D⊂R]^{+2(•+)} bisradical complex and D^{+(•+)}, a process that affords the [D⊂R]^{+3(•+)} trisradical complex. These results provide supportive evidence for the two possible SET pathways towards the final product of the molecular recognition, as illustrated in Scheme 9 (FIG. 54).

### 6.4 Investigation of the [2]Catenane Model Compound

In order to unravel the superstructure and investigate the properties of the bisradical complex, we synthesized a [2]catenane (Cat•6PF₆) as a model compound for detailed studies. Although the bisradical complex was originally detected during the molecular recognition process, the combination of R^{2(•+)}, D^{+(•+)} and CoCp₂ constitutes a complicated supramolecular system (FIG. 26), involving seven kinds of BIPY-based chemical species and seven reversible processes. In contrast, the [2]catenane—as a result of the mechanical bond^{23,24} between two component rings—is a well-defined molecular system (FIG. 26), including no more than two kinds of BIPY-based species and only one process. The simplicity of this molecular system allows us to obtain entirely a bisradical-state [2]catenane, namely Cat^{2(•+)}, by controlling the amount of CoCp₂, which is helpful for the investigation of its properties. In addition, Cat^{2(•+)} has proven to be crystallizable, enabling the direct observation of its structure.

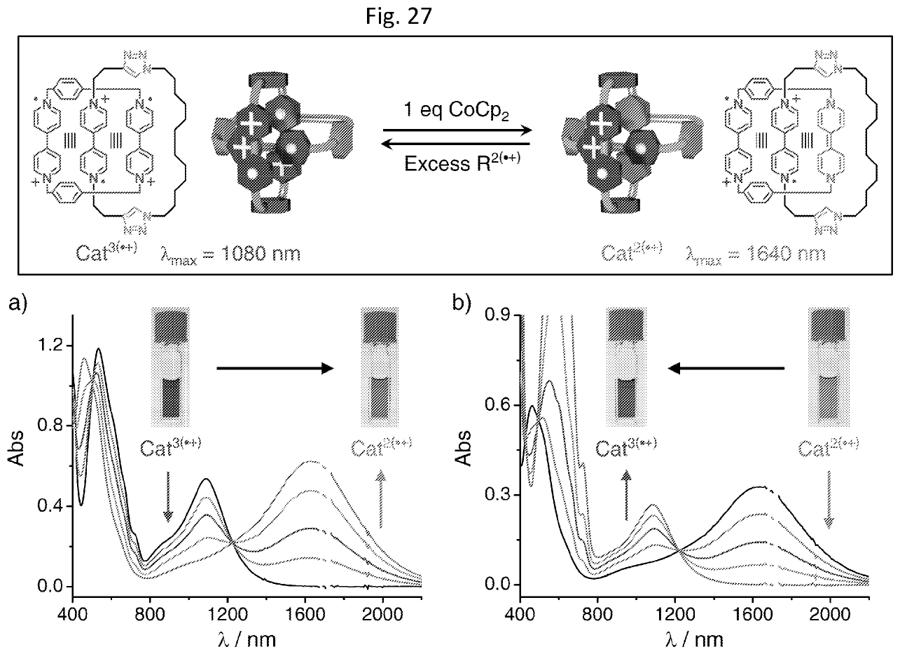

The trisradical tricationic state of the [2]catenane, Cat^{3(•+)}, was prepared by stirring the MeCN solution of Cat•6PF₆ (150 μM) with excess of activated Zn dust for 30 min. Thereafter, the solid was filtered out to afford a purple solution. This solution of Cat^{3(•+)} underwent titration with 1.0 eq CoCp₂, and the evolution of the UV/Vis/NIR spectra was recorded. During the titration, the decrease of the absorption band at 1080 nm was observed (FIG. 27a), accompanied by increase of the absorption at 1640 nm, with isobestic points at 506 nm and 1219 nm. This result indicates the quantitative transformation from Cat^{3(•+)} to Cat^{2(•+)}. The narrow band gap (less than 0.62 eV, deduced²⁵ from λₒₙₛₑₜ≥2000 nm) of Cat^{2(•+)} is probably because of the combination of radical-pairing and donor-acceptor interactions.

Furthermore, when excess of R^{2(•+)} was introduced into the solution of Cat^{2(•+)}, as expected, the absorption band at 1640 nm was found (FIG. 27b) to decay, along with the recovery of the absorption band at 1080 nm. This spectral change, indicating that Cat^{2(•+)} was oxidized back to Cat^{3(•+)}, confirms the ability of the bisradical state to undergo SET with R^{2(•+)}.

In order to grow the single crystal of Cat^{2(•+)}, 4.0 eq CoCp₂ was added into the MeCN solution of Cat•6PF₆ (1.0 mM). Thereafter, slow vapor diffusion of Et₂O into the MeCN solution was allowed to occur in the N₂-filled glovebox over one week, affording black crystals suitable for X-ray crystallographic analysis.

Methods. A suitable crystal was selected and mounted on a MITIGEN holder with Paratone oil on a XtaLAB Synergy R, DW system, HyPix diffractometer. The crystal was kept at 100.01(10) K during data collection. Using Olex2 (Ref²⁶), the structure was solved with the ShelXT structure solution program²⁷ using Intrinsic Phasing and refined with the XL refinement package²⁸ using Least Squares minimisation.

Crystal data for C₇₆H₈₄CoF₁₈N₁₂P₃ (M=1659.39). Tetragonal, space group P4̅c2 (no. 116), a=16.09100(10) Å, c=30.6313(4) Å, V=7931.06(14)Å³, Z=4, T=100.01(10)K, μ(CuK_{α})=3.075 mm⁻¹, D_{calc}=1.390 g cm⁻³, 54393 reflections measured (5.492≤2Θ≤157.412), 8323 unique (Rᵢₙₜ=0.0342, R_{sigma}=0.0215) which were used in all calculations. The final R₁ was 0.0851 (I>2σ(I)) and wR₂ was 0.2472 (all data).

Refinement Details. Distance restraints were imposed on the carbon chain and disordered PF₆⁻ anions. The enhanced rigid-bond restraint (SHELX keyword RIGU) was applied²⁹ on the carbon chain and disordered PF₆⁻ anions. Restraints on similar amplitudes separated by less than 1.7 Å. were also imposed on the disordered carbon chain.

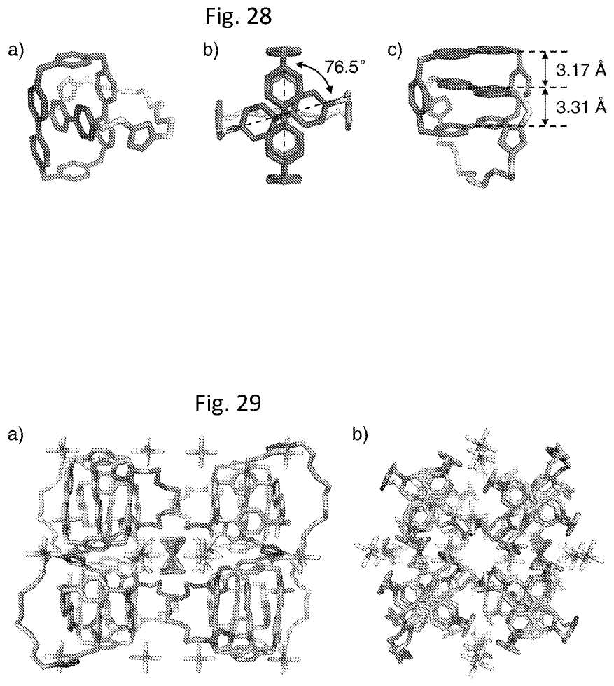

The solid-state (super)structures are shown in FIGS. 28 and 29.

TABLE 6
Comparison between the two possible resonance isomers of the bisradical binding state

| Resonant Isomerism | 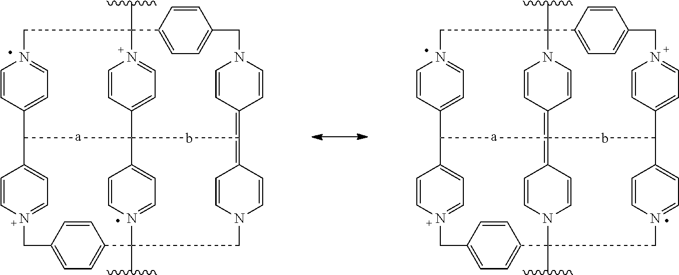 | |
|---|---|---|
| Magnetic Property | Diamagnetic | Paramagnetic |
| Structure | Asymmetric | Symmetric |
| | (a ≠ b) | (a = b) |
| Interactions | Radical-Pairing | Donor-Acceptor |
| | Donor-Acceptor | |
| Contribution | Major | Minor |

Cat²(•+), which involves the through-space delocalization of two electrons over three BIPY units, can be presented as the hybridization of two possible resonance isomers. These two isomers exhibit different structures and properties, as summarized in Table 6. According to 1) the narrow bandgap estimated from the NIR absorption, 2) the diamagnetic nature indicated by the silent EPR signal, and 3) the uneven distances between adjacent BIPY units observed in the X-ray crystallographic data, we determined the asymmetric structure as the major isomer of Cat^{2(•+)} wherein the BIPY⁽⁰⁾ neutral unit is located on one side rather than in the middle. The structural feature of Cat^{2(•+)} can be generalized to its non-interlocked counterpart, i.e., a noncovalent bisradical complex.

### 7. Molecular Recognition Promoted by Various Chemical Initiators

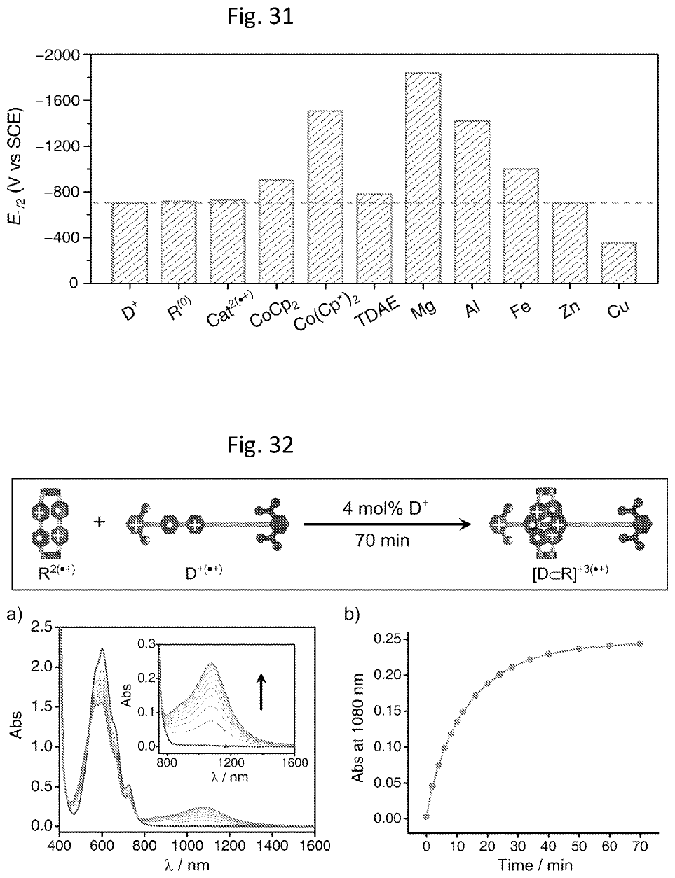

The mechanism of electron catalysis prompted us to screen a variety of chemical initiators (FIG. 30) for promoting the molecular recognition between R^{2(•+)} and D^{+(•+)}. Firstly, a small amount of either D⁺ and R⁽⁰⁾—the reduction product of R^{2(•+)} or D^{+(•+)}, respectively—can be used directly to initiate the molecular recognition process. Secondly, the bisradical-state [2]catenane Cat^{2(•+)} exhibits a similar effect. Thirdly, some other reductants without BIPY structural units-such as bis(cyclopentadienyl) cobalt(II) (cobaltocene, CoCp₂), bis(pentamethylcyclopentadienyl) cobalt(II), Co(Cp*)₂ for short, and tetrakis(dimethylamino)ethylene (TDAE)—have also shown their potency to trigger the molecular recognition process. Finally, active metals, including magnesium (Mg), aluminium (Al), iron (Fe) and zinc (Zn), can serve as heterogenous initiators, although copper (Cu) has no such effect because of the insufficient reducing power. The related experimental data are shown in FIGS. 32-38. In summary, the reducing power (FIG. 31) required for triggering this molecular recognition process is around −700 mV. It means that any reagents—with 1) the appropriate reduction potential that is lower than, or comparable to, this value and 2) the ability to transfer an electron at a reasonable rate—are eligible chemical initiators.

### 8. Molecular Recognition Initiated or Controlled by Electricity

The mechanism of electron catalysis renders it possible to initiate or control the molecular recognition between R^{2(•+)} and D^{+(•+)} using electricity. For this purpose, the electrolysis of the mixture of R^{2(•+)} and D^{+(•+)} was performed in a divided or undivided cell. These two kinds of electrochemical set-ups⁹ have shown distinct effects on the molecular recognition process. Specifically, the divided-cell approach, which enables the permanent injection of catalytic amounts of electrons, is analogous to the introduction of chemical initiators. In contrast, during the continuous electrolysis in an undivided cell, electrons are simultaneously injected into and withdrawn from the solution, allowing temporal control of the molecular recognition process.

### 8.1 Electrochemically Initiated Molecular Recognition in a Divided Cell

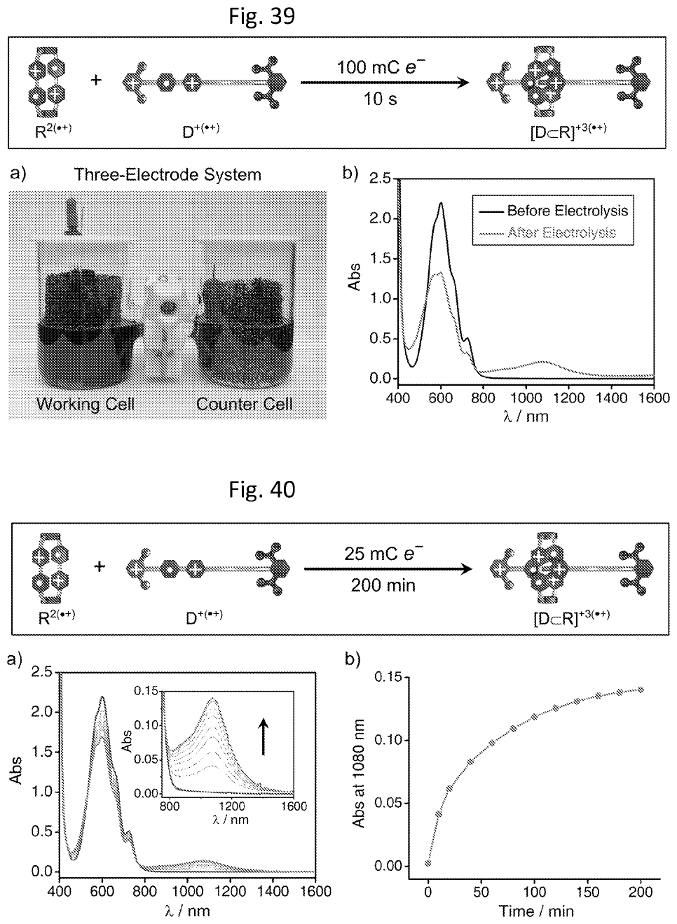

Electrochemically initiated molecular recognition between R^{2(•+)} and D^{+(•+)} was performed in a N₂-filled glovebox using a divided cell. A three-electrode system was employed, which included a reticular vitreous carbon (RVC) working electrode, an RVC counter electrode and an Ag/AgCl reference electrode. The working cell (FIG. 39a, left) was filled with a MeCN solution (50 mL) containing R^{2(•+)} (150 μM), D^{+(•+)} (150 μM) and TBAPF₆ (0.05 μM), while the counter cell (FIG. 39a, right) was filled with a MeCN solution (50 mL) of excess of (trimethylammonium methyl)ferrocene hexafluoro-phosphate, which serves as the sacrificial electron donor. The two cells were separated by an ionic exchange membrane and held together by a clamp. The whole apparatus was connected to a Gamry multipurpose instrument (Reference 600) interfaced to a PC. The experimental parameters were instructed using the software of Gamry Framework Version 6.30 under the chronocoulometry mode. In order to inject a certain number (catalytic amount) of electrons into the host-guest system, the solutions were electrolysed under stirring at a constant potential of −0.65 V for only a few seconds before switching off the electricity.

This approach of using divided-cell electrolysis has displayed a similar catalytic effect to that involving the introduction of chemical initiators. The injection of 100 mC (14 mol %) of electrons allows (FIG. 39b) the molecular recognition between R^{2(•+)} and D^{+(•+)} to be completed instantaneously (within 10 s). In contrast, by injecting 25 mC (3.5 mol %) of electrons and then switching off the electricity, the molecular recognition proceeds (FIG. 40) at a relatively slow rate, allowing the process to be monitored by UV/Vis/NIR spectroscopy.

### 8.2 Electrochemically Controlled Molecular Recognition in an Undivided Cell

Electrochemically controlled molecular recognition between R^{2(•+)} and D^{+(•+)} was conducted in a N₂-filled glovebox using an IKA® Electrasyn 2.0 Device. The set-up was an undivided cell in which both the cathode and anode are reticular vitreous carbon electrodes. In a typical procedure, a MeCN solution (9 mL) containing R^{2(•+)} (150 μM), D^{+(•+)} (150 μM) and TBAPF₆ (0.05 μM) underwent electrolysis for 3 min at a constant current (e.g., 1.0 mA) and a constant stirring rate (e.g., 300 rpm). Subsequently, the electricity was switched off and the solution was allowed to stand for 3 min. The overall process consisted of three on/off cycles. Molecular recognition during the intermittent electrolysis was monitored by sampling the solution every 3 min and recording its UV/Vis/NIR spectrum. In order to reveal the influence of current intensity and stirring rate on the kinetics of molecular recognition, two arrays of electrolysis experiments were carried out. In the first array, the stirring rate was held at 300 rpm, while the current was set to 0.5, 1.0 or 2.0 mA. In the second array, the current was held at 1.0 mA, while the stirring rate was set to 200, 300 or 400 rpm.

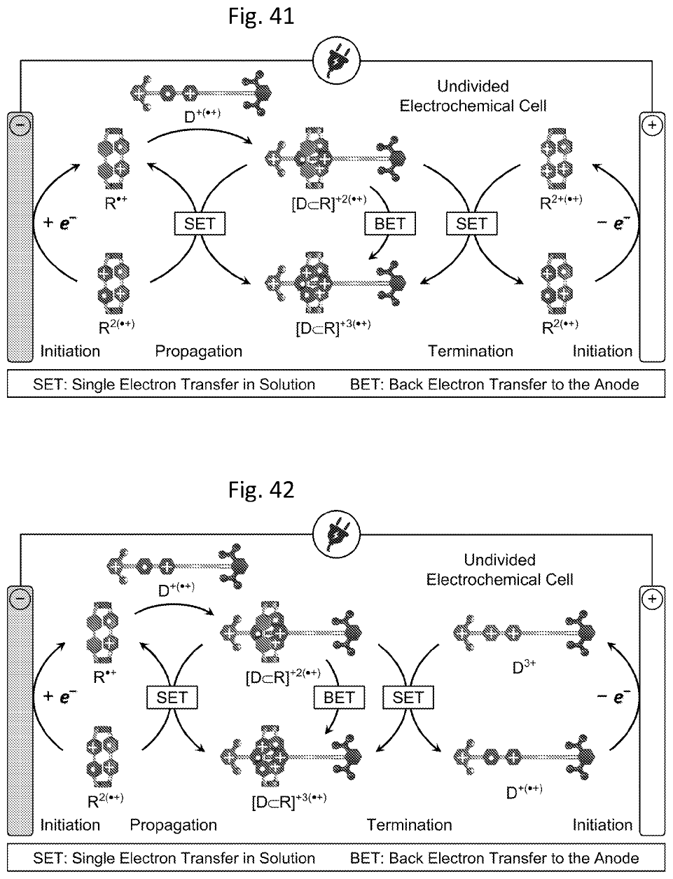

Four possible pathways involving the molecular recognition between R^{2(•+)} and D^{+(•+)} during the undivided-cell electrolysis are illustrated in FIGS. 41-44. All the pathways follow a similar mechanism, while the difference lies in which component in the system is reduced/oxidized at the interface of the cathode/anode, respectively, in the initiating step.

In a combined view of the above four possible pathways, the electrochemically controlled molecular recognition between R^{2(•+)} and D^{+(•+)} occurs through an “initiation-propagation-termination” process, which can be summarized as below. Note that the electron at the cathode and the anode is labelled as e_{c}⁻ and eₐ⁻, respectively.

### Initiation:

$${R}^{2 ( \cdot + )} + {e}_{c}^{-} \to {R}^{\cdot +} {D}^{+ ( \cdot + )} + {e}_{c}^{-} \to {D}^{+}$$

$${R}^{2 ( \cdot + )} - {e}_{a}^{-} \to {R}^{2 + ( \cdot + )} {D}^{+ ( \cdot + )} + {e}_{a}^{-} \to {D}^{3 +}$$

### Propagation:

$${R}^{\cdot +} + {D}^{+ ( \cdot + )} ⇌ {[ D ⊂ R ]}^{+ 2 ( \cdot + )} {D}^{+} + {R}^{2 ( \cdot + )} ⇌ {[ D ⊂ R ]}^{+ 2 ( \cdot + )}$$

$${[ D ⊂ R ]}^{+ 2 ( \cdot + )} + {R}^{2 ( \cdot + )} ⇌ {[ D ⊂ R ]}^{+ 3 ( \cdot + )} + {R}^{\cdot +}$$

$${[ D ⊂ R ]}^{+ 2 ( \cdot + )} + {D}^{+ ( \cdot + )} ⇌ {[ D ⊂ R ]}^{+ 3 ( \cdot + )} + {D}^{\cdot +}$$

### Termination:

$${R}^{\cdot +} + {R}^{2 + ( \cdot + )} \to {R}^{2 ( \cdot + )} {D}^{+} + {D}^{3 +} \to {D}^{+ ( \cdot + )}$$

$${[ D ⊂ R ]}^{+ 2 ( \cdot + )} + {R}^{2 + ( \cdot + )} \to {[ D ⊂ R ]}^{+ 3 ( \cdot + )} + {R}^{2 ( \cdot + )}$$

$${[ D ⊂ R ]}^{+ 2 ( \cdot + )} + {D}^{+} \to {[ D ⊂ R ]}^{+ 3 ( \cdot + )} + {D}^{+ ( \cdot + )}$$

$${R}^{2 + ( \cdot + )} + {e}_{c}^{-} \to {R}^{2 ( \cdot + )} {D}^{3 +} + {e}_{c}^{-} \to {D}^{+ ( \cdot + )}$$

$${[ D ⊂ R ]}^{+ 2 ( \cdot + )} - {e}_{a}^{-} \to {[ D ⊂ R ]}^{+ 3 ( \cdot + )}$$

The overall process:

$${R}^{2 ( \cdot + )} + {D}^{+ ( \cdot + )} ⇌ {[ D ⊂ R ]}^{+ 3 ( \cdot + )}$$

These equations cover the possible processes at the beginning stage of electrolysis. When the electrochemically controlled molecular recognition has proceeded for a period of time, the [D⊂R]^{+3(•+)} trisradical complex is formed and accumulated. At this time, besides the starting materials R^{2(•+)} and D^{+(•+)}, the complex can also accept/lose an electron at the cathode/anode, rendering the pathways more complicated.

TABLE 7
The calculated conversions of electrochemically controlled
molecular recognition under different conditions

| | | Conversion/% | | |
|---|---|---|---|---|
| Conditions | | 3 min | 6 min | 9 min |
| 300 rpm | 0.5 mA | 0.6 | 1.4 | 3.2 |
| | 1.0 mA | 3.5 | 6.9 | 11.2 |
| | 2.0 mA | 10.8 | 18.0 | 24.0 |
| 1.0 mA | 200 rpm | 18.6 | 29.1 | 31.3 |
| | 300 rpm | 3.5 | 6.9 | 11.2 |
| | 400 rpm | 1.0 | 3.6 | 4.5 |

TABLE 8
The estimated values of Faradic efficiencies of electrochemically
controlled molecular recognition under different conditions

| | | Faradic Efficiency/% | | |
|---|---|---|---|---|
| Conditions | | 3 min | 6 min | 9 min |
| 300 rpm | 0.5 mA | 0.9 | 1.0 | 1.6 |
| | 1.0 mA | 2.5 | 2.5 | 2.7 |
| | 2.0 mA | 3.9 | 3.3 | 2.9 |
| 1.0 mA | 200 rpm | 13.5 | 10.5 | 7.5 |
| | 300 rpm | 2.5 | 2.5 | 2.7 |
| | 400 rpm | 0.7 | 1.3 | 1.1 |

The conversions and Faradic efficiencies of electrochemically controlled molecular recognition are estimated and summarized in Tables 7 and 8. As expected, the conversion can be improved with the increased current intensity and is lowered with the increased stirring rate. In the case of 1.0 mA, 200 rpm, the conversion after 9 min electrolysis is 31.3%, a value close to the conversion at the thermodynamic equilibrium (37.4%).

In order to understand the values of Faradic efficiency, we probed the electrolysis process in the undivided cell. When a BIPY^{•+} radical cation, whether in R^{2(•+)} or D^{+(•+)}, picks up an electron from the cathode to form a BIPY^{(O)} unit, there can be three possible subsequent pathways: In Pathway 1, the species bearing the BIPY^{(O)} unit moves quickly to the anode and returns an electron, or encounters the BIPY²⁺ unit generated from the anodic oxidation. As a result, BIPY^{•+} is restored before molecular recognition has happened. The Faradic efficiency is 0.

In Pathway 2, a [D⊂R]^{+2(•+)} bisradical complex is generated, which deposits an electron at the anode or undergo single electron transfer with a BIPY²⁺ unit to generate the final product, i.e., the [D⊂R]^{+3(•+)} trisradical complex. As a result, the transfer of one electron from cathode to anode contributes to one round of molecular recognition. This process is a stoichiometric one, and the Faradic efficiency is 100%.

In Pathway 3, a [D⊂R]^{+2(•+)} bisradical complex is generated, which transfers an electron to BIPY^{•+}, thereby affording a [D⊂R]^{+3(•+)} trisradical complex and a BIPY²⁺ unit. Subsequently, this newly formed BIPY⁽⁰⁾ can propagate molecular recognition in a “chain reaction” manner. As a result, one electron can induce more than one round of molecular recognition. This process is justified as catalysis, and the Faradic efficiency is larger than 100%.

In a practical setting, the electrolysis experiments result in a combination of these three pathways. The distributions of them rely on the mass transport of chemical species. Since the solution is stirred during the process, convection should be the major form of mass transport and play an important role in regulating the lifetime of catalytic intermediates. Pathway 1 is the major one when convection is very fast, so that the lifetime of BIPY⁽⁰⁾ is not long enough to induce molecular recognition before returning to BIPY^{•-}. In contrast, Pathway 3 is dominant when convection is very slow, so that BIPY⁽⁰⁾ has sufficient time to sustain many cycles of molecular recognition. Pathway 2 is somewhere in between.

All the values of Faradic efficiencies (Table 8) are lower than 100%, an observation which indicates that Pathway 1 plays a dominant role under the conditions that we have employed. The Faradic efficiency could be improved by lowering the stirring rate.

### 9. Theoretical Analyses of the Molecular Recognition Processes

### 9.1 Chemically Initiated Molecular Recognition

From the theoretical perspective, when the electron is introduced chemically by a reductant (Red), it will trigger the molecular recognition. In order to simplify the expression, we only consider the case in which R^{2(•+)} accepts an electron from the reductant, and so the process can be described as:

### Initiation:

$${R}^{2 ( \cdot + )} + Red ⇌ {R}^{\cdot +} + {Red}^{+}$$

### Propagation:

$${R}^{\cdot +} + {D}^{+ ( \cdot + )} ⇌ {[ D ⊂ R ]}^{+ 2 ( \cdot + )}$$

$${[ D ⊂ R ]}^{+ 2 ( \cdot + )} + {R}^{2 ( \cdot + )} ⇌ {[ D ⊂ R ]}^{+ 3 ( \cdot + )} + {R}^{\cdot +}$$

The overall process:

$${R}^{2 ( \cdot + )} + {D}^{+ ( \cdot + )} {⇌}^{{K}_{eq}} {[ D ⊂ R ]}^{+ 3 ( \cdot + )}$$

The overall process is a catalysis by the electron, that is to say, molecular recognition proceeds faster than it would without the reductant, but its equilibrium constant remains the same. An arbitrarily small (catalytic) amount of reductant can in principle facilitate an arbitrarily large number of conversions from R^{2(•+)} and D^{+(•+)} to the [D⊂R]^{+3(•+)} trisradical complex, i.e., bring the molecular recognition to its equilibrium which, based on the free energy differences, is essentially to completion.

### 9.2 Electrochemically Controlled Molecular Recognition

The electrochemically controlled molecular recognition contains two possible processes, depending on the convection rates of intermediates during the electrolysis in an undivided cell. If the convection is slow, the number of catalytic cycles prior to termination will be large and the system can be considered to operate in a catalytic regime. This process is identical with the chemically initiated molecular recognition in terms of the theoretical consideration described above. If the convection is fast, the number of catalytic cycles will be small and the system can be considered to operate in a stoichiometric regime, where the transfer of one electron from cathode to anode promotes the formation of only one supramolecular complex.

In order to describe the stoichiometric process, let us only consider the case in which R^{2(•+)} accepts an electron from the cathode. During the event, R^{2(•+)} picks up an electron at the cathode (e_{c}⁻) and binds with D^{+(•+)} to form a [D⊂R]^{+2(•+)} bisradical complex as the intermediate, which deposits the electron at the anode (eₐ⁻) to afford the final product, i.e., the [D⊂R]^{+3(•+)} trisradical complex:

$${R}^{2 ( \cdot + )} + {D}^{+ ( \cdot + )} + {e}_{c}^{-} ⇌ {R}^{\cdot +} + {D}^{+ ( \cdot + )}$$

$${R}^{\cdot +} + {D}^{+ ( \cdot + )} ⇌ {[ D ⊂ R ]}^{+ 2 ( \cdot + )}$$

$${[ D ⊂ R ]}^{+ 2 ( \cdot + )} ⇌ {[ D ⊂ R ]}^{+ 3 ( \cdot + )} + {e}_{a}^{-}$$

The overall process is the combination of these three steps:

$${e}_{c}^{-} + {R}^{2 ( \cdot + )} + {D}^{+ ( \cdot + )} {⇌}^{{K}_{eq} {e}^{FV / RT}} {[ D ⊂ R ]}^{+ 3 ( \cdot + )} {e}_{a}^{-} \qquad I$$

where F is Faraday constant, V is the applied voltage, R is the universal gas constant and T is temperature.

According to this equation, the assembly of R^{2(•+)} and D^{+(•+)} into the [D⊂R]^{+3(•+)} trisradical complex is coupled to the electron transfer from cathode to anode, and so the steady-state position of this supramolecular system is influenced by the voltage. Therefore, we must conclude that the effect of electrons supplied electrochemically in this stochiometric process is not catalysis, even though the rate of molecular recognition is much larger than that in the absence of an electron supply.

Furthermore, according to trajectory thermodynamics³³, we should also consider another pair of forward and microscopic reverse processes.

$${[ D ⊂ R ]}^{+ 3 ( \cdot + )} + {e}_{c}^{-} ⇌ {[ D ⊂ R ]}^{+ 2 ( \cdot + )}$$

$${[ D ⊂ R ]}^{+ 2 ( \cdot + )} ⇌ {R}^{\cdot +} + {D}^{+ ( \cdot + )}$$

$${R}^{\cdot +} + {D}^{+ ( \cdot + )} ⇌ {R}^{2 ( \cdot + )} + {D}^{+ ( \cdot + )} + {e}_{a}^{-}$$

$${e}_{c}^{-} + {[ D ⊂ R ]}^{+ 3 ( \cdot + )} {⇌}^{{K}_{eq}^{- 1} {e}^{FV / RT}} {R}^{2 ( \cdot + )} + {D}^{+ ( \cdot + )} + {e}_{a}^{-} \qquad II$$

Combining the process I and II, the steady-state concentration ratio among R^{2(•+)}, D^{+(•+)} and the [D⊂R]^{+3(•+)} trisradical complex is determined by a kinetically weighted average of two voltage-constrained equilibrium constants:

$${\frac{[ {[ D ⊂ R ]}^{+ 3 ( \cdot + )} ]}{[ {R}^{2 ( \cdot + )} ] [ {D}^{+ ( \cdot + )} ]} \vert}_{SS} = \frac{{aK}_{eq} {e}^{FV / RT} + {bK}_{eq} {e}^{- FV / RT}}{a + b} = {K}_{eq} {\underbrace{[ \frac{{e}^{FV / RT} q + {e}^{- FV / RT}}{q + 1} ]}}_{}$$

In this equation, a and b are weighting coefficients of the process I and II, respectively, and their ratio is defined as q, whose value in general depends on the applied voltage. [symbol] is the kinetic asymmetry³⁴ parameter that is bounded between e^{FV/RT} and e^{-FV/RT}.

Therefore, the stoichiometric process during the electrolysis has two features. Firstly, there is a one-to-one correspondence between the number of (i) electrons accepted by BIPY^{•+} units at the cathode and transferred subsequently to the anode and (ii) complexes formed during the process. Secondly, the energy released by transfer of an electron from the cathode to the anode can, depending on the kinetic asymmetry of the process, shift the overall equilibrium of the supramolecular system.

### 10. REFERENCES

1 Barnes, J. C., Juricek, M., Vermeulen, N. A., Dale, E. J. & Stoddart, J. F. Synthesis of Ex″Box cyclophanes. J. Org. Chem. 78, 11962-11969 (2013).
2 Pezzato, C. et al. An efficient artificial molecular pump. Tetrahedron 73, 4849-4857 (2017).
3 Cheng, C. et al. Energetically demanding transport in a supramolecular assembly. J. Am. Chem. Soc. 136, 14702-14705 (2014).
4 Coskun, A., Saha, S., Aprahamian, I. & Stoddart, J. F. A reverse donor-acceptor bistable [2]catenane. Org. Lett. 10, 3187-3190 (2008).
5 Li, H. et al. Mechanical bond formation by radical templation. Angew. Chem. Int. Ed. 49, 8260-8265 (2010).
6 Wang, Y. et al. Symbiotic control in mechanical bond formation. Angew. Chem. Int. Ed. 55, 12387-12392 (2016).
7 Lipke, M. C. et al. Size-matched radical multivalency. J. Am. Chem. Soc. 139, 3986-3998 (2017).
8 Pezzato, C. et al. Controlling dual molecular pumps electrochemically. Angew. Chem. Int. Ed. 57, 9325-9329 (2018).
9 Kingston, C. et al. A survival guide for the “electro-curious”. Acc. Chem. Res. 53, 72-83 (2020).
10 Qiu, Y. et al. A precise polyrotaxane synthesizer. Science 368, 1247-1253 (2020).
11 Trabolsi, A. et al. Radically enhanced molecular recognition. Nat. Chem. 2, 42-49 (2010).
12 Fahrenbach, A. C. et al. Solution-phase mechanistic study and solid-state structure of a tris(bipyridinium radical cation) inclusion complex. J Am. Chem. Soc. 134, 3061-3072 (2012).
13 Cheng, C. et al. Influence of constitution and charge on radical pairing interactions in tris-radical tricationic complexes. J Am. Chem. Soc. 138, 8288-8300 (2016).
14 Goren, Z. & Willner, I. Photochemical and chemical reduction of vicinal dibromides via phase transfer of 4,4′-bipyridinium radical: The role of radical disproportionation. J. Am. Chem. Soc. 105, 7764-7765 (1983).
15 Maidan, R., Goren, Z., Becker, J. Y. & Willner, I. Application of multielectron charge relays in chemical and photochemical debromination processes. The role of induced disproportionation of NN-dioctyl-4,4′-bipyridinium radical cation in two-phase systems. J Am. Chem. Soc. 106, 6217-6222 (1984).
16 Mohammad, M. Methyl viologen neutral MV. 1. Preparation and some properties. J. Org. Chem. 52, 2779-2782 (1987).
17 Frasconi, M. et al. Redox control of the binding modes of an organic receptor. J Am. Chem. Soc. 137, 11057-11068 (2015).
18 Zhao, Y. & Truhlar, D. G. The M06 suite of density functionals for main group thermochemistry, thermochemical kinetics, noncovalent interactions, excited states, and transition elements: Two new functionals and systematic testing of four M06-class functionals and 12 other functionals. Theor. Chem. Acc. 120, 215-241 (2007).
19 Jaguar, Version 10.6 (Schrodinger, Inc., New York, 2019). Tannor, D. J. et al. Accurate first principles calculation of molecular charge distributions and solvation energies from ab initio quantum mechanics and continuum dielectric theory. J Am. Chem. Soc. 116, 11875-11882 (1994).
21 Cheng, M.-J., Nielsen, R. J., Tahir-Kheli, J. & Goddard III, W. A. The magnetic and electronic structure of vanadyl pyrophosphate from density functional theory. Phys. Chem. Chem. Phys. 13, 9831-9838 (2011).
22 Wertz, D. H. Relationship between the gas-phase entropies of molecules and their entropies of solvation in water and 1-octanol. J. Am. Chem. Soc. 102, 5316-5322 (1980).
23 Bruns, C. J. & Stoddart, J. F. The Nature of the Mechanical Bond: From Molecules to Machines. (Wiley, 2016).
24 Stoddart, J. F. Mechanically interlocked molecules (MIMs)—Molecular shuttles, switches, and machines (Nobel lecture). Angew. Chem. Int. Ed 56, 11094-11125 (2017).
25 Guha, S. et al. Electronically regulated thermally and light-gated electron transfer from anions to naphthalenediimides. J Am. Chem. Soc. 133, 15256-15259 (2011).
26 Dolomanov, O. V., Bourhis, L. J., Gildea, R. J., Howard, J. A. K. & Puschmann, H. OLEX2: A complete structure solution, refinement and analysis program. J. Appl. Crystallogr. 42, 339-341 (2009).
27 Sheldrick, G. M. SHFLXT—Integrated space-group and crystal-structure determination. Acta Cryst. A71, 3-8 (2015).
28 Sheldrick, G. M. A short history of SHELX. Acta Cryst. A64, 112-122 (2008).
29 Thorn, A., Dittrich, B. & Sheldrick, G. M. Enhanced rigid-bond restraints. Acta Cryst. A68, 448-451 (2012).
30 Burkholder, C., Dolbier, W. R. & Medebielle, M. Tetrakis(dimethylamino)ethylene as a useful reductant of some bromodifluoromethyl heterocycles. Application to the synthesis of new gem-difluorinated heteroarylated compounds. J Org. Chem. 63, 5385-5394 (1998).
31 Gennett, T., Milner, D. F. & Weaver, M. J. Role of solvent reorganization dynamics in electron-transfer processes. Theory-experiment comparisons for electrochemical and homogeneous electron exchange involving metallocene redox couples. J Phys. Chem. 89, 2787-2794 (1985).
32 Larson, R. C. & Iwamoto, R. T. Solvent effects on the polarographic reduction of metal ions. I. Benzonitrile-acetonitrile. J Am. Chem. Soc. 82, 3239-3244 (1960).
33 Astumian, R. D. et al. Non-equilibrium kinetics and trajectory thermodynamics of synthetic molecular pumps. Mater. Chem. Front. 4, 1304-1314 (2020).
34 Astumian, R. D. Kinetic asymmetry allows macromolecular catalysts to drive an information ratchet. Nat. Commun. 10, 3837 (2019).

1. A system for electron catalyzed molecular recognition, the system comprising:
an electron source for providing an electron;
a redox-active substrate capable of accepting the electron from the electron source;
a catalytic intermediate formed noncovalently from the substrate and a second molecule, wherein the energy barrier for forming the catalytic intermediate is decreased by the redox-active substrate accepting the electron from the electron source; and,
optionally, a final product.

2. The system of claim 1, wherein the electron source is an undivided electrochemical cell.

3. The system of claim 1, wherein the electron source is a divided electrochemical cell.

4. The system of claim 1, wherein the electron source is a chemical initiator.

5. The system of claim 4, wherein the chemical initiator is a homogeneous chemical initiator.

6. The system of claim 4, wherein the chemical initiator is a heterogeneous chemical initiator.

7. The system of claim 1, wherein the catalytic intermediate is a bisradical host-guest complex.

8. The system of claim 1 comprising the final product, wherein the final product is a trisradical host-guest complex.

9. The system of claim 8, wherein the catalytic intermediate is a bisradical host-guest complex.

10. The system of claim 1, wherein the redox-active substrate comprises a marcrocyclic host ring R or a dumbbell-shaped guest D and the catalytic intermediate is a bisradical host-guest complex formed from the macrocyclic host ring and the dumbbell-shaped guest.

11. The system of claim 10, wherein the macrocyclic host ring comprises two bipyridinium (BIPY) units and/or wherein the dumbbell-shaped guest comprises a bipyridinium (BIPY) unit and a cationic terminus.

12. The system of claim 10 comprising the final product, wherein the final product is a trisradical host-guest complex formed from the macrocyclic host ring and the dumbbell-shaped guest.

13. A method for electron catalyzed molecular recognition, the method comprising:
providing, with an electron source, an electron to a redox-active substrate capable of accepting the electron from the electron source; and
forming noncovalently a catalytic intermediate from the redox-active substrate and a second molecule, wherein the energy barrier for forming the catalytic intermediate is decreased by the redox-active substrate accepting the electron from the electron source; and,
optionally, forming a final product.

14. The method of claim 13, wherein the electron source is an undivided electrochemical cell.

15. The method of claim 13, wherein the electron source is a divided electrochemical cell.

16. The method of claim 13, wherein the electron source is a chemical initiator.

17. The method of claim 16, wherein the chemical initiator is a homogeneous chemical initiator.

18. The method of claim 16, wherein the chemical initiator is a heterogeneous chemical initiator.

19. The method of claim 13, wherein the catalytic intermediate is a bisradical host-guest complex.

20. The method of claim 13 comprising forming the final product, wherein the final product is a trisradical host-guest complex.

21. The method of claim 20, wherein the catalytic intermediate is a bisradical host-guest complex.

22. The method of claim 13, wherein the redox-active substrates comprises a marcrocyclic host ring R or a dumbbell-shaped guest D and the catalytic intermediate is a bisradical host-guest complex formed from the macrocyclic host ring and the dumbbell-shaped guest.

23. The method of claim 22, wherein the macrocyclic host ring comprises two bipyridinium (BIPY) units and/or wherein the dumbbell-shaped guest comprises a bipyridinium (BIPY) unit and a cationic terminus.

24. The method of claim 22 comprising forming the final product, wherein the final product is a trisradical host-guest complex formed from the macrocyclic host ring and the dumbbell-shaped guest.
